(19) United States
(12) Patent Application Publication
(10) Pub. No.: US 2004/0083060 A1
(43) Pub. Date: Apr. 29, 2004

(54) Method of operating a computer system to perform a discrete substructural analysis

(76) Inventors: Dennis Church, Commugny (CH); Jacques Colinge, Neydens (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/399,329

(22) PCT Filed: Oct. 16, 2001
(86) PCT No.: PCT/EP01/11955

(30) Foreign Application Priority Data
Oct. 17, 2000 (EP) 00309114.7

(51) Int. Cl. G06F 19/00; G01N 33/48; G01N 33/50

(52) U.S. Cl. 702/19

(57) ABSTRACT

The invention provides a method of operating a computer system, and a corresponding computer system, for performing a discrete substructural analysis. First, a database of molecular structures is accessed. The database is searchable by molecular structure information and biological and/or chemical properties. In said database, a set of molecules is identified that have a given biological and/or chemical property. Fragments of the molecules in said subset are then determined, and a score value is calculated for each fragment, indicating the contribution of the respective fragment to said given biological and/or chemical property. Finally, a reiteration process is performed by analyzing the determined fragments and calculated scores values, whereby first at least one fragment is selected that has a score value indicating high contribution to said biological and/or chemical property, and then the steps of accessing, identifying, determining and calculating are repeated. Fragments may be any structural subunit of the molecules. The biological and/or chemical properties include biochemical, pharmacological, toxicological, pesticidal, herbicidal and catalytic properties. The invention is preferably used for DNA backsequencing or drug discovery. Preferred embodiments include an reiteration process that increases the fragment size in each iteration, the use of generiy substructures, and an annealing process that glues fragments together.

The present invention relates to a computer system and a method of operating same, capable of performing a discrete substructural analysis. The analysis allows for performing a computer implemented identification of molecules having certain properties such as biological and/or chemical activity. The computer controlled discrete substructural analysis can be used in drug discovery or in other fields where the identification of biologically, pharmacologically, toxicologically, pesticidally, herbicidally, catalytically etc. active compounds is of interest.

Advances within the field of, e.g., medicinal chemistry depend upon the identification of biologically active molecules. In many instances, research programs are targeted towards synthesis of small organic molecules which will interact with a known enzyme or receptor target, in order to produce a desired pharmacological effect. Such compounds may, at least in part, mimic or inhibit the activity of a known, naturally occurring substance, but are intended to provide a more potent and/or more selective action. Compounds arising from this type of research may incorporate certain structural features of the relevant naturally occurring substances.

Research programs may also be based on naturally occurring compounds found as a result of screening sources available in nature, for example soil samples or plant extracts. Active compounds discovered in this manner may be useful leads for a program of synthetic chemistry.

In recent years the pressure to identify new and useful biologically active molecules has increased, and in consequence, new methods of generating lead compounds have been developed. Two developments have been of particular importance in this respect, namely combinatorial chemistry and high throughput screening (HTS).

Combinatorial chemistry employs robotic or manual techniques to carry out a multiplicity of small scale chemical reactions each using a different combination of reagents, simultaneously or ‘in parallel’, thereby generating large numbers of diverse chemical entities for screening. The collection of compounds generated by this method is known as a ‘library’. Libraries for generating novel chemical leads are usually as diverse as possible. However, in certain circumstances libraries may be biased or targeted towards a particular pharmacological target, or focussed on a particular chemical area, by selecting reagents intended to introduce specific structural features in the final, compounds.

High throughput screening involves the use of biochemical assays to rapidly test the in vitro activity of large numbers of chemical compounds against one or more biological targets. This method is ideal for screening the large libraries of compounds generated by combinatorial chemistry.

Despite the undoubted advantages of combinatorial chemistry and HTS in generating new lead structures, there are some drawbacks with these methods. A high proportion of the compounds in unbiased combinatorial libraries have no useful activity. Discovery of useful leads therefore relies on chance and/or the number of compounds tested. Targeted libraries may have a higher proportion of active compounds, but are dependent upon selection criteria and may even fail to provide optimum compounds. Furthermore both techniques require considerable resources and experimental capacity.

The chance or probability of finding an active molecule in a given compound set can be increased either by increasing the total number of compounds tested (i.e. the size of the sets) or by increasing the proportion of active compounds in the same set. It can be shown that increasing the proportion of active compounds in a compound collection is more effective for increasing the probability of finding an active molecule than simply increasing the total number of compounds that are tested. The former approach reduces the number of compounds which need to be made and tested and is therefore also more favourable in terms of the resources required e.g. for finding biologically active molecules.

A substructural analysis as an approach to the problem of drug design is disclosed in Richard D. Cramer III. et al., J. Med. Chem., 17 (1974), pages 553 to 535. It is described that the biological activity of a molecule, or any other of its properties, must be accounted for by a combination of contributions from its structural components (substructures) and their intra- and intermolecular interactions. The contribution of a given substructure to the probability of activity can be obtained from data on previously tested compounds containing that substructure. A first step is to prepare a substructure “experience table” summarizing the available data. A “Substructure Activity Frequency” (SAF) is defined for each substructure as the ratio of the number of active compounds containing that substructure to the number of tested compounds containing that substructure. The SAF is said to represent the contribution which that substructure can make to the probability of a compound being active. Then, for each compound the arithmetic mean of the SAF values of the substructures present in that compound is computed.

While this prior art technique allows for ranking compounds by their mean SAF values, obtaining such a value requires the calculation of an arithmetic mean of the SAF values of each substructure that is present in the compound. Moreover, the SAF values required for this calculation are the result of a previous computation that involves the evaluation of each substructure in each one of the tested molecules. This approach therefore leads to a significant computational overhead that prevents this technique from being applied to larger data sets that are presently available and that could be used as source of information for performing a molecular structure analysis. The Cramer method, however, fails to actually estimate the true contribution that a substructure makes to activity.

There are therefore a number of further prior art techniques in the field of chemical structure analysis.

EP 938 055 A describes a method for developing quantitative structure activity relationships on the basis of data generated from high throughput screening, by identifying structural characteristics which render compounds ‘active’. The method is designed to establish a statistical model for biologically active compounds which first associates various chemical descriptors to a given collection of compounds and then, by using a sub-group of compounds of known biological activity, trains the model to predict whether a new compound would be biologically active or not.

Sheridan and Kearsley, J. Chem. Inf. Comput. Sci., 35 (1995), pages 310-320 describe the use of genetic algorithms to select a sub-set of fragments for use in constructing a combinatorial library. This method involves generating a population of molecules from a sub-set of molecular fragments, and calculating a score for each molecule, based on specified descriptors (e.g. atom pair or topological torsion) using either similarity probe or trend vector methods. Further populations are generated using the genetic algorithm, and scored. The results provide a list of fragments that occur in maximally scoring molecules, which can be used as the basis for constructing a combinatorial library.

WO 99/26901A1 discloses a method of designing chemical substances such as molecules. A compound consists of a scaffold and number of sites. The method starts with selecting candidate elements for the sites and creating a predictive designed array PAD. An example of a PAD consists of a number of virtual compounds fulfilling certain combinational conditions. These compounds are then synthesized and tested for a biological activity. Then, an algorithm is performed for predicting the overall biological activity of those compounds which have not been synthesized. For this purpose, property contribution values for the candidate elements are calculated, representing the respective contribution of each of the individual elements to the activity. Further, the average contribution of each substituent group at a particular site to the biological activity is calculated. An example of how to calculate such contribution is given.

H. Gao et al., J. Chem. Inf. Comput. Sci. (39) 1999, 164-168 is an article describing the application of a QSAR (quantitative structure-activity relationship) technique to a drug discovery problem. After biologically active compounds are selected, their biological activity is optimized. Since QSAR is based on a hypothetical relationship between biological activity and molecular structures, the technique is concerned with identifying structural characteristics that render compounds active and predicting active and inactive analogs.

WO 00/41060 A1 discloses a method for correlating substance activities with structural features for substances. The term “feature” relates to atoms and bonds of a structure that matches a pattern. In a first step, the members of a substance set are determined that satisfy given structural feature and property constraints. Then, for each activity category, the substances that fall in said category are designated. After partitioning the set of substances among several activity categories, the expected activity for any subset is calculated and, for each structural feature a set of activity-property-feature bit vectors are constructed which designate the numbers of substances that contain said feature and are in said activity category. The document relates to biological activities and is also concerned with drug discovery.

U.S. Pat. No. 6,185,506 B1 discloses a method for selecting an optimally diverse library of small molecules based on validated molecular structural descriptors. Multiple literature data sets are used which contain a variety of chemical structures and associated activities. Activity may be biological and chemical activity. The technique is described in the context of pharmacological drugs. Further, a method for selecting a subset of product molecules is disclosed for all possible product molecules which could be created in a combinatorial synthesis from specified reactant molecules and common core molecules. In the section describing the background art, reference is made to biologically specific libraries which have been designed based on knowledge about geometric arrangements of structural fragments abstracted from molecular structures known to have activity. It is disclosed as being absolutely necessary to use a smaller rationally designed screening library which still retains the diversity of the combinatorially accessible compounds.

WO 00/49539 A1 discloses a method for screening a set of molecules for identifying sets of molecular features that are likely to correlate with a specified activity. The term feature relates to chemical substructures. A set of molecules is grouped according to their molecular structure as characterized by a set of descriptors. Then, the groups that represent a high level of activity are identified and the most common substructures among the molecules in the groups are found which may reasonably be correlated to the observed activity level. A data set is established that represents those molecules from an initial data set that include the common subset of features. The technique is described as taking the form of a computer-based system for the automated analysis of a data set.

U.S. Pat. No. 5,463,564 discloses a computer-based method of automatically generating compounds by robotically synthesizing and analyzing a plurality of chemical compounds. The process is performed iteratively and aims at generating chemical entities with defined activity properties. A directed diversity chemical library is synthesized that comprises a plurality of chemical compounds. Structure-activity data are obtained by robotically analyzing the synthesized compounds. A number of databases are disclosed that each include a field indicating a rating factor assigned to the respective compound. The rating factor is assigned to each compound based on how closely the compound's activity matches a desired activity.

The aforementioned methods are either “predictive” models or still fail to sufficiently improve the generation of active leads and increase the probability of finding active compounds within a given set of compounds. Further, the conventional techniques are incapable of satisfying the need for an increased number and quality of molecule hits and leads that enter the development pipeline.

It is therefore the object of the invention to provide a method of operating a computer system, and a corresponding computer system, capable of increasing the chance of discovering novel, biologically and/or chemically active molecules.

This object is solved by the invention as claimed in the independent claims.

Preferred embodiments are defined in the dependent claims.

One advantage of the invention is that a computer system and an operation method is provided that allow for increasing the proportion of active compounds in a given set of chemical entities where said entities are not already known to have the desired activity. This is performed by applying knowledge-based techniques to identify novel hit and lead series, notably by building systems for conducting a computationally driven molecule discovery.

Another advantage of this invention is that by means of analysing a database that is searchable by molecular structures and biological and/or chemical properties, costly experiments are avoided. The discovery process of the invention can therefore be rationalised which will in turn lead to a less expensive drug discovery.

Further, the invention advantageously allows for performing discovery processes more rapidly so that molecules having certain desired properties can be identified in a shorter time compared with the prior art methods.

Further, the invention is in particular advantageous in the field of biochemistry. In the past, DNA sequencing, and in particular genome sequencing, has provided comprehensive databases of amino acid sequences that can be used as starting point when performing the invention. Then, the invention allows for identifying known and/or orphan ligands and/or orphan ligand-receptor pairs by predicting a peptide sequence on the basis of results obtained with a list of structures analyzed for biologically active chemical determinants. After identification in a database and expression, the peptide sequences can be tested by a biochemical assay. Thus, the invention advantageously permits to deduce biological structures by comparison with a list of chemical molecules, for which activity on a certain target had been determined, and thus provides an identification (backsequencing) technique.

The invention will now be described in more detail with reference to the figure drawings, in which:

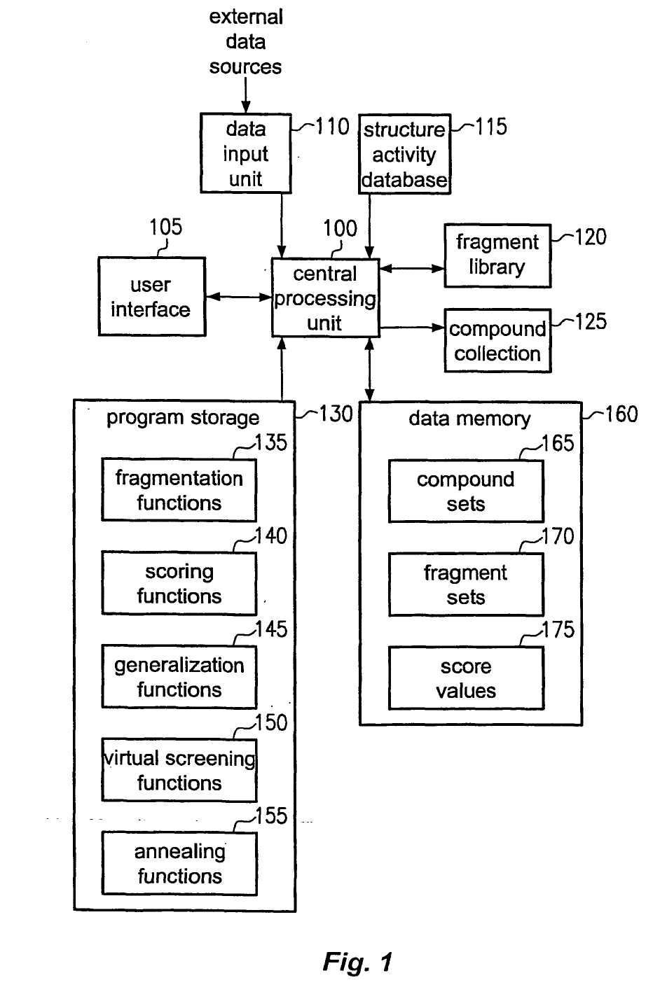

FIG. 1 is a block diagram illustrating the computer system according to a preferred embodiment of the invention;

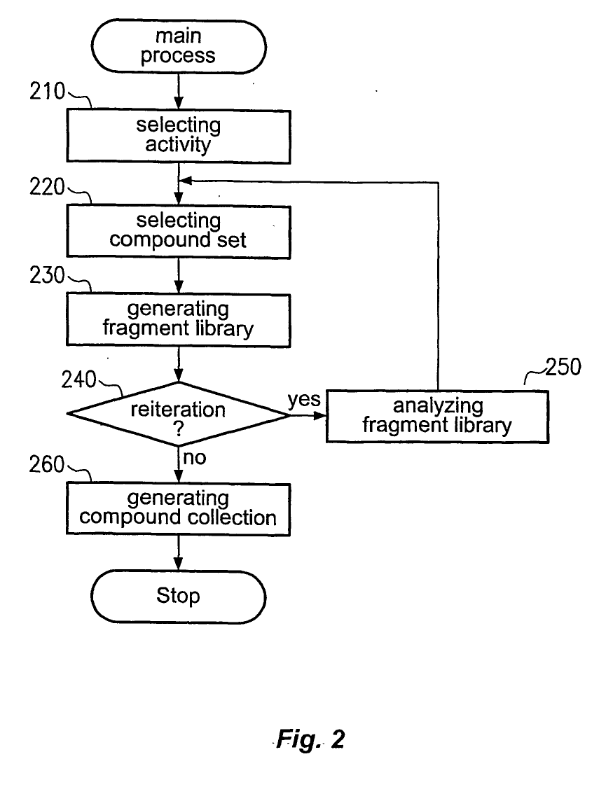

FIG. 2 is a flowchart illustrating the main process of performing a discrete structural analysis according to a preferred embodiment of the invention;

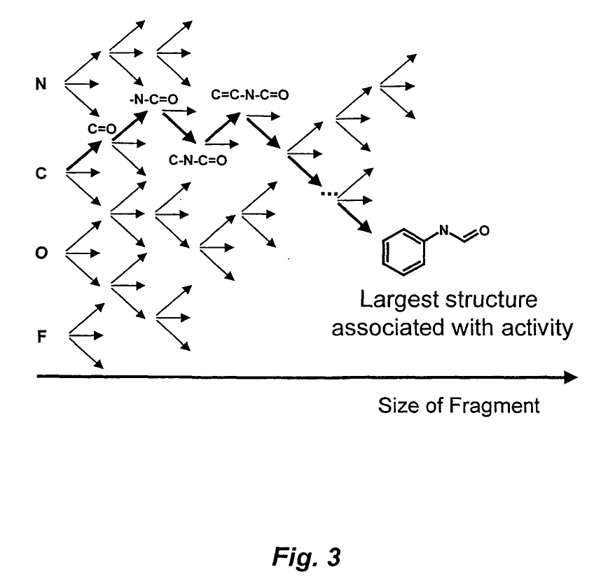

FIG. 3 is a schematic drawing illustrating the reiteration process of the invention;

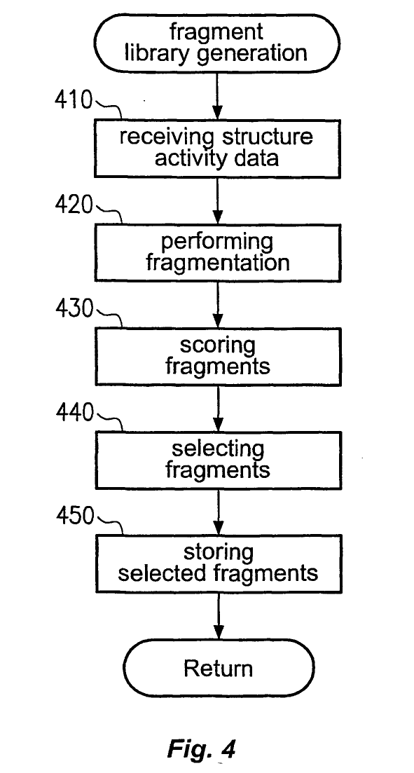

FIG. 4 is a flowchart illustrating the process of generating a fragment library according to a preferred embodiment of the present invention;

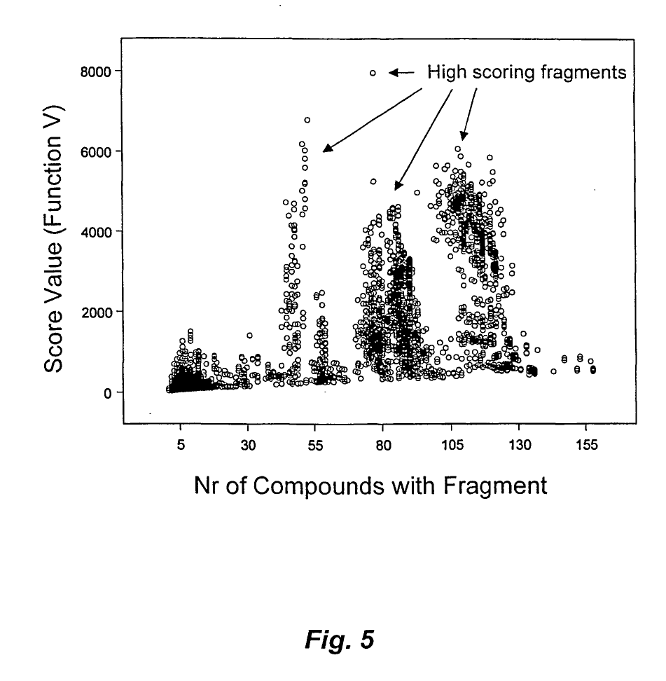

FIG. 5 is a graph illustrating how fragments can be selected based on the calculated score values;

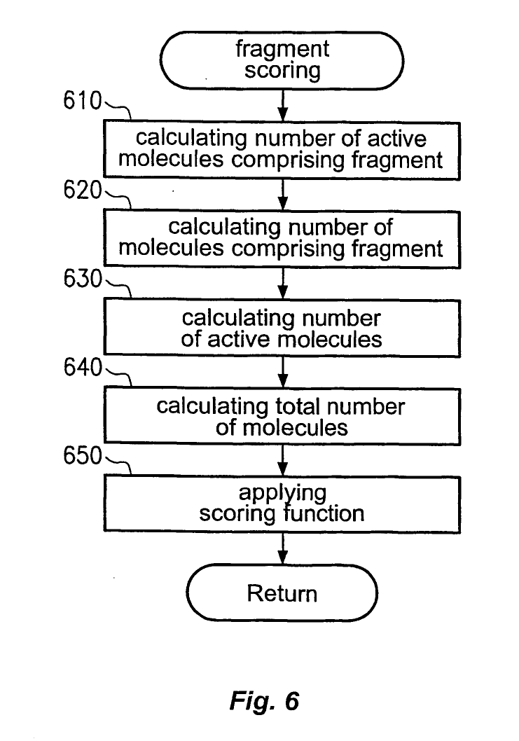

FIG. 6 is a flowchart illustrating the process of calculating a score value for a fragment, according to a preferred embodiment of the present invention;

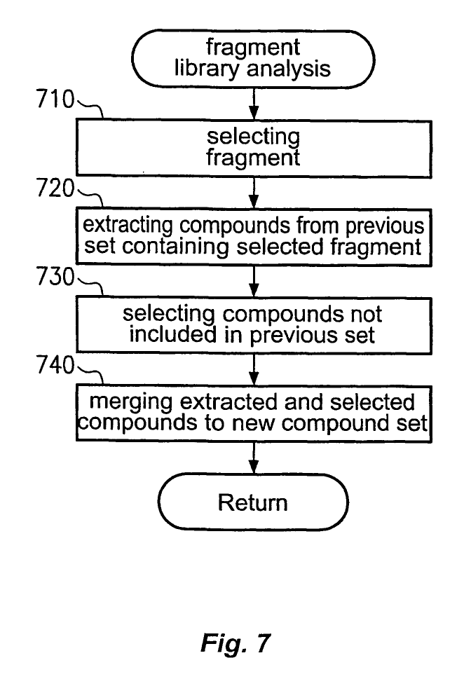

FIG. 7 is a flowchart illustrating the process of analysing the fragment library when performing a reiteration;

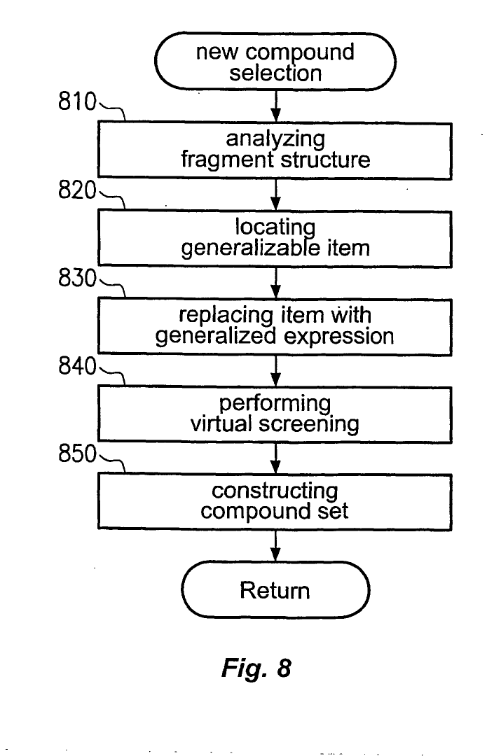

FIG. 8 is a flowchart illustrating the process of selecting a new compound by using generic substructures;

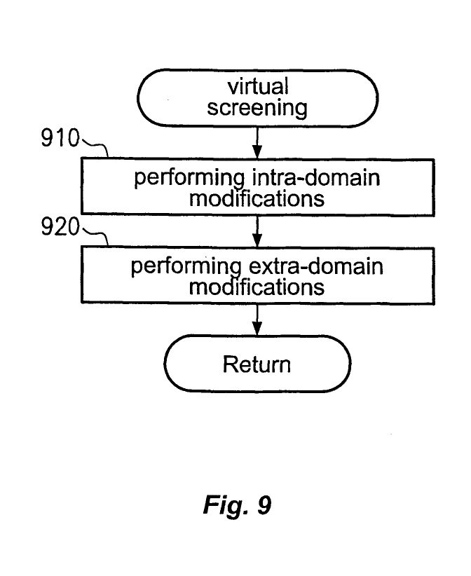

FIG. 9 is a flowchart illustrating the process of generating substructures for use in virtual screening;

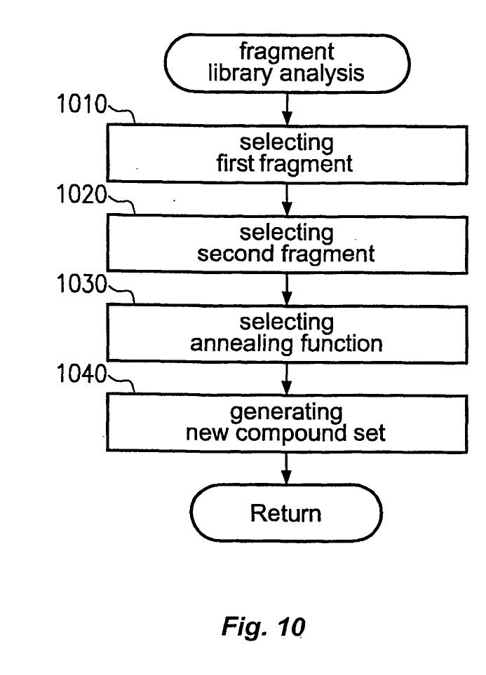

FIG. 10 is a flowchart illustrating the process of analysing the fragment library when performing a reiteration, applying the annealing technique according to a preferred embodiment of the invention;

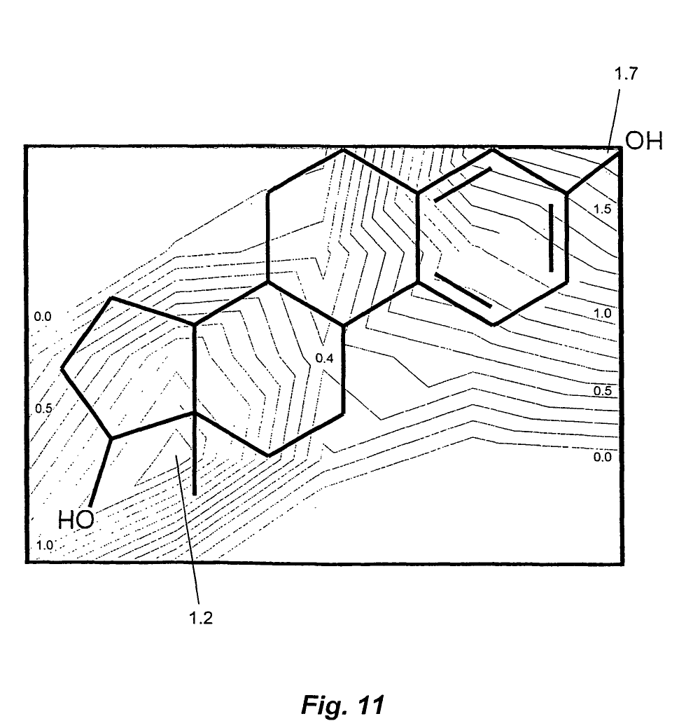

FIG. 11 is an example of a relative contribution map for illustrating the annealing technique applied in-the process of FIG. 10;

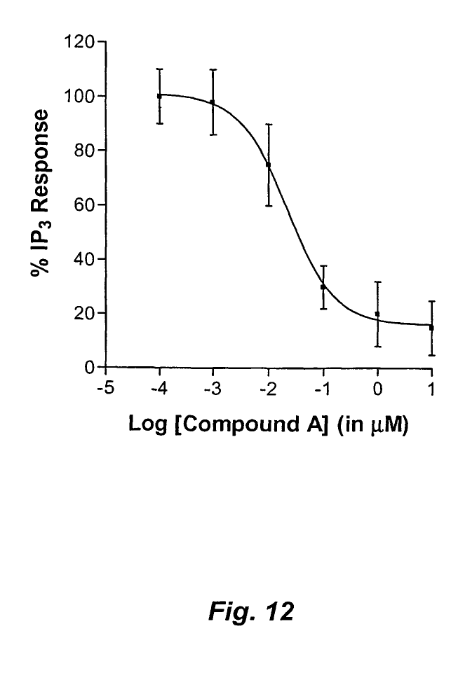

FIG. 12 is a graph illustrating the effect of a compound on receptor-mediated inositol triphosphate generation;

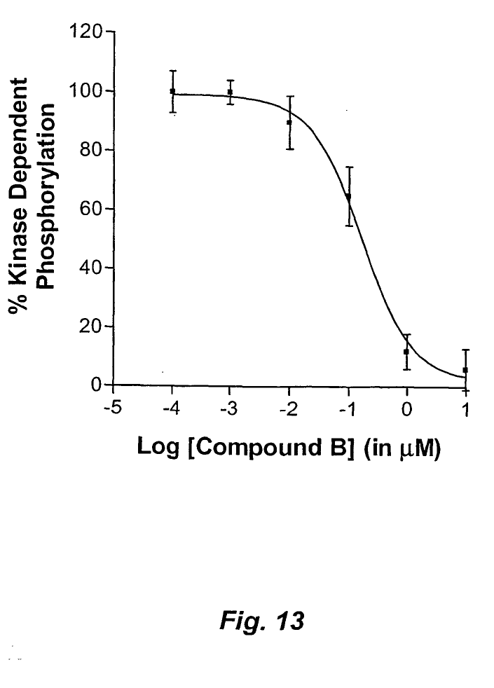

FIG. 13 is a graph illustrating the effect of a compound on kinase-dependent protein phosphorylation;

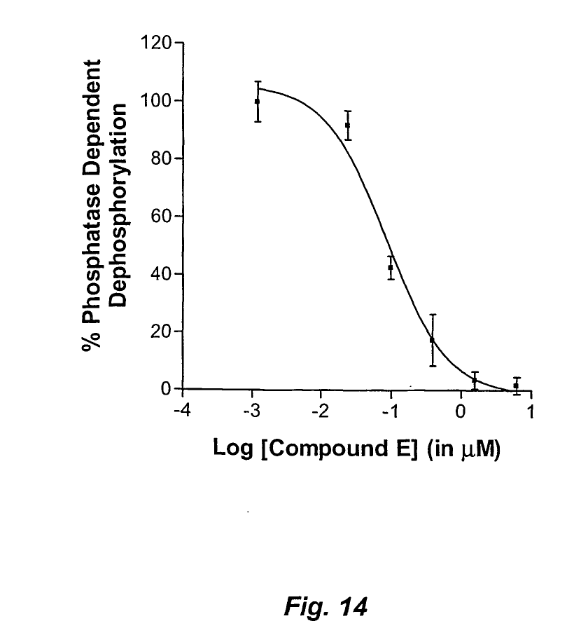

FIG. 14 is a graph illustrating the effect of a compound on phosphatase-dependent protein dephosphorylation;

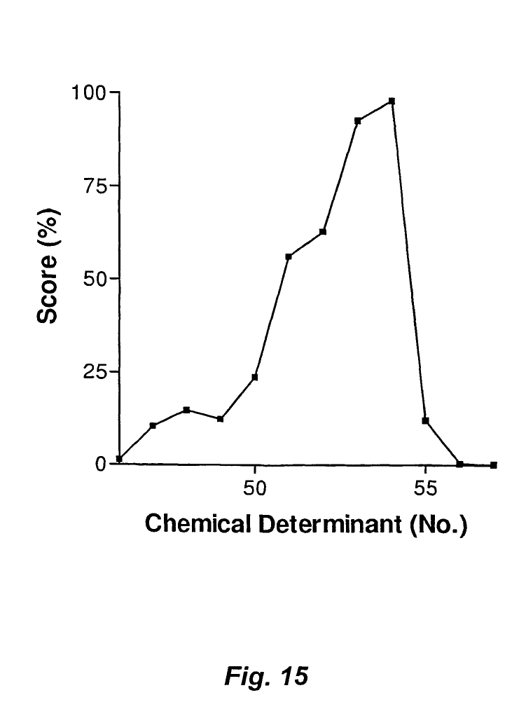

FIG. 15 is a graph showing relative contribution information by plotting determinants versus their respective score values; and

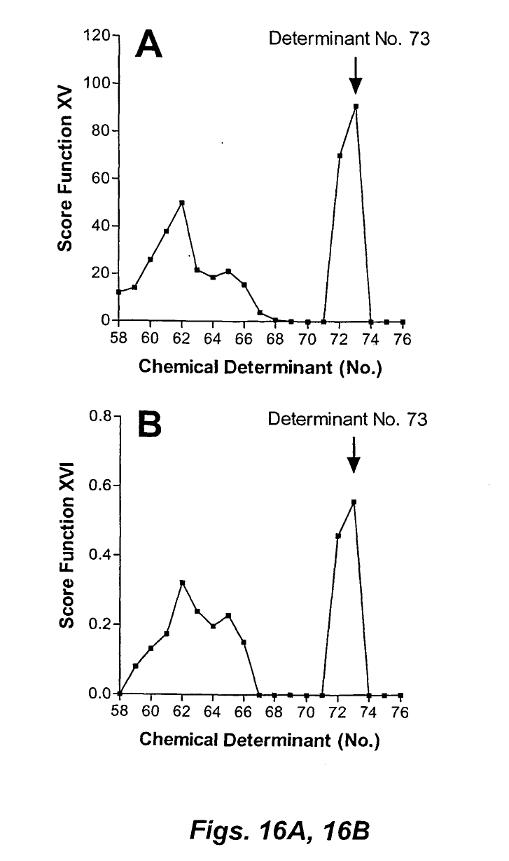

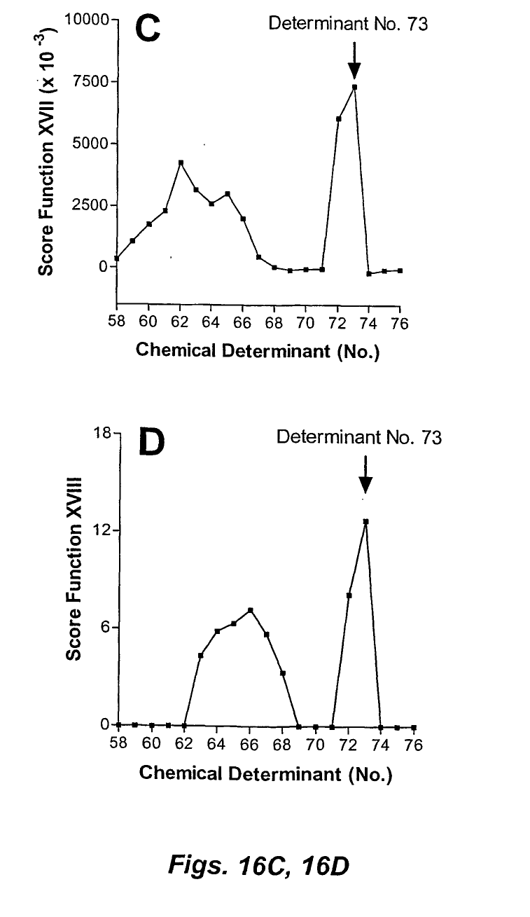

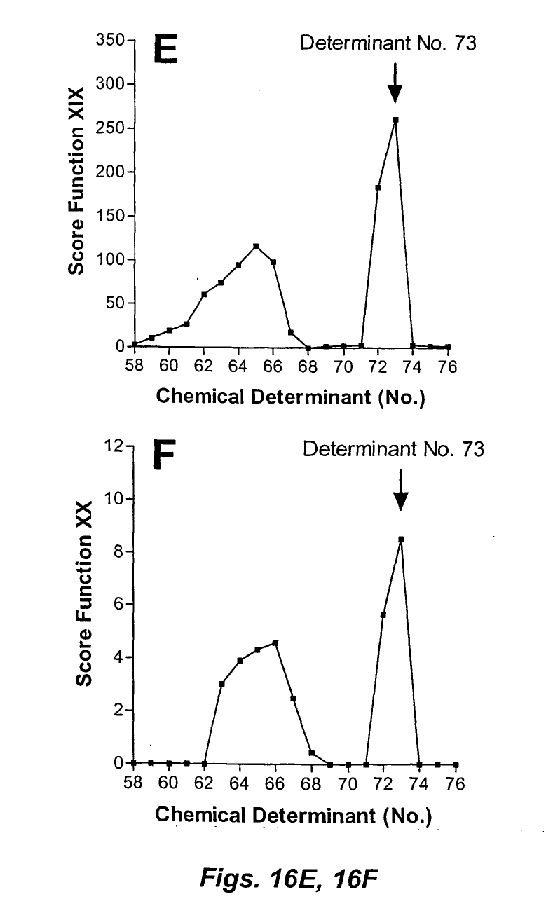

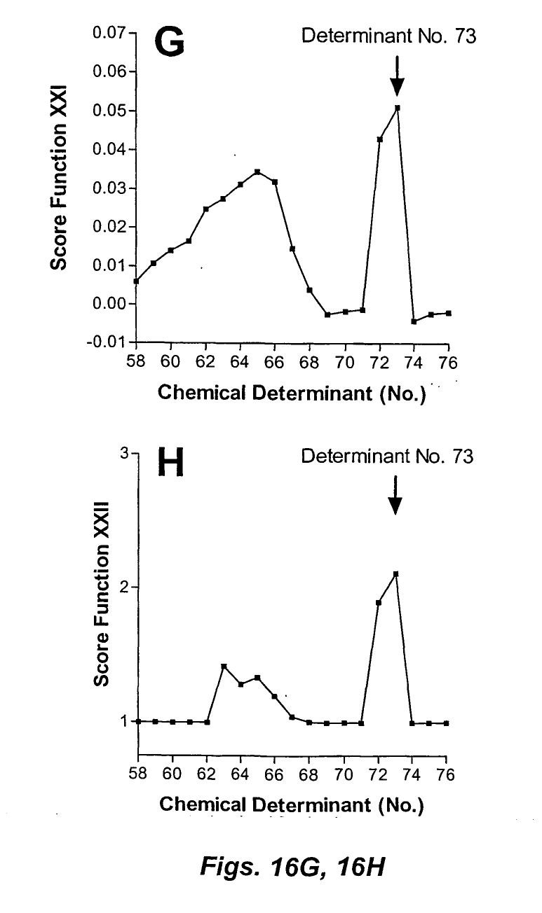

FIGS. 16A-H are further relative contribution diagrams demonstrating the equivalence of score functions.

The present invention will now be described in more detail. Further, preferred embodiments of the invention will be discussed with reference to the accompanying figure drawings. Moreover, a number of examples are given of how the invention can be applied in numerous fields of compound discovery.

According to the invention, a computer system is operated to perform a discrete substructural analysis. A database of molecular structures is accessed. The database is searchable by molecular information and biological and/or chemical properties. Molecular structure information is any information suitable for determining the molecular structure of a molecule. Biological and/or chemical properties include biochemical, pharmacological, toxicological, pesticidal, herbicidal, and catalytic properties.

Using the database, the technique according to the present invention identifies a subset of molecules having a given biological and/or chemical property. In said subset, fragments of the molecules are then determined. The term “fragment” relates to any structural subunit of a molecule, including simple functional groups, two-dimensional substructures and families thereof, simple atoms or bonds, and any assembly of structural descriptors in the two-dimensional or three-dimensional molecular space. It will be appreciated by those of ordinary skill in the art that a fragment may be a molecular substructure that is of no known meaning in conventional chemistry.

After the molecular structures in the subset are broken down into fragments, a score value is calculated for each fragment indicating the contribution of the respective fragment to the given biological and/or chemical property. That is, the invention allows for assigning a score value to fragments based on existing knowledge with respect to biological and/or chemical properties of molecules. In the following description, a molecule, structure or sub-structure is said to be “active” if it has the given property. A molecule, structure or sub-structure not being active is said to be “inactive”. Thus, the present invention provides a sub-structural analysis based on discrete biological and/or chemical property information. The main process of the invention is therefore hereafter called Discrete Substructural Analysis (DSA).

Since according to the invention, fragments are associated with score values indicating their contribution to a given biological and/or chemical property, fragments can be considered as chemical determinants responsible for a given biological and/or chemical outcome. The identification of fragments is accomplished by following a set of logical rules (algorithm), which are inherent to the DSA process itself. In this context, the score value is itself a function of:

(a) the prevalence of the chemical determinant in the subset of active molecules, and

(b) the prevalence of the same said determinant in the entire list of compounds under consideration.

On the basis of this definition, the method then identifies one or more local extrema of the score function, whose corresponding chemical determinants represent full or partial chemical solutions to the desired biological outcome. Finding the largest possible values that the score function can achieve in any given data set is equivalent to identifying the chemical determinants contained within subsets of the most potent biologically active molecules which have the lowest probability of occurring by chance in the same subsets.

The invention will now be described with reference to the figure drawings, and in particular referring to FIG. 1. FIG. 1 depicts a preferred embodiment of a computer system according to the invention. The computer system comprises a central data processing unit 100 that can be controlled by user interface means 105. Units 100 and 105 may be any computer system such as a work station or personal computer. Preferably, the computer system is a multiprocessor system running a multi-tasking operating system.

The central processing unit 100 is connected to a program storage 130 that stores executable program code including instructions for performing the DSA process according to the invention. These instructions include fragmentation functions 135 for breaking down molecular structures into fragments, score functions 140 for calculating score values, generalisation functions 145 (to retrieve isomers for instance) for locating generalisable items in fragment structures and replacing these items with generalised expressions thereby generating generic substructures, virtual screening functions 150 for performing a virtual screening, and annealing functions 155 for performing the fragment annealing process of the invention. Details on the individual functions and the processors performed by the central processing unit 100 in executing these functions will be described in more detail below.

The central processing 100 is further connected to a structure activity database, or compound activity list, 115 to receive molecular structure information and biological and/or chemical property information. This information can likewise be received from a data input unit 110 that allows for accessing external data sources.

By accessing units 110 and/or 115, the subset of molecular structures may be obtained for example from any available source such as a proprietary or public database which is searchable by substructure and/or biological properties. Public databases include but are not limited to those available under the following names: MDDR, Pharmaprojects, Merck Index, SciFinder, Derwent. The subset of molecules may also be obtained by synthesising and testing compounds. The molecules will generally comprise complete compounds, but they may also themselves be molecular fragments. For any given biological or chemical property, the subset contains compounds which do not possess the said property, for example compounds which are not active (or fall below a given activity threshold) as well as compounds which do possess the said property, for example, compounds which exhibit the desired activity (i.e. have activity above a given threshold). All non-active compounds are relevant, and are therefore analysed.

After accessing the internal or external data and performing the DSA process using functions stored in program storage 130, the central processing unit 100 stores a fragment library 120 that contains the determined fragments of the molecules together with associated score values.

In one preferred embodiment of the present invention, the fragment library 120 is the result of the main process according to the invention. The fragment library 120 can then be used for instance by chemical and biological scientists or engineers as a source of valuable information that is usable in any subsequent discovery process.

In another preferred embodiment, the fragment library 120 is an intermediate result of the main process of the invention and can therefore be stored in a volatile as well as a non-volatile memory. The fragment library 120 according to this embodiment may be read by the central processing unit 100 in executing further functions stored in the program storage 130 for generating a compound collection 125.

The compound collection 125 is a collection of molecules that have been revealed by the process of the invention as having the desired biological and/or chemical property or not. The molecules of the compound collection 125 may either be already known or may be hypothetical structures that have not been synthesised before. In any case, the molecules of the compound collection 125 are the result of evaluating the score values assigned to the fragments according to the discrete substructural analysis.

As can be seen from FIG. 1, the central processing unit 100 is further connected to a data memory 160 that stores compound sets 165, fragment sets 170 and score values 175. The data memory 160 is provided for storing data that is used for storing input parameters when invoking the functions 135-155, or for storing return values of these functions.

Referring now to FIG. 2 which illustrates a preferred embodiment of the main DSA process, the operator of the computer system depicted in FIG. 1 first selects an activity in step 210. As mentioned above, activity means any biological and/or chemical property including biochemical, pharmacological, toxicologically, pesticidal, herbicidal, catalytic properties. Moreover when using the invention for identifying orphan ligands, an activity may be a given effect on a protein of interest (typically binding).

In the present specification, reference to a particular property, such as biological activity, may, unless the context dictates otherwise, be extrapolated to other types of biological and/or chemical property. Furthermore, for the avoidance of doubt, the terms ‘compound’, ‘molecule’ and ‘molecular structure’ may all encompass molecular substructures as well as complete compounds, according to the context.

After an activity has been selected in step 210, a compound set 125 is selected in step 220. The selected compound set is a set of molecules that are to be examined to learn which fragments contribute to the selected activity. As will be described in more detail below, the compound set selected in step 220 includes molecules that are known to be active and molecules that are known to be inactive.

Once an activity and a compound set have been selected, the process continues with the generation of a fragment library 120 in step 230. The process of generating the fragment library can be described as a process of weighting the efficacy of molecular fragments, within a subset of known structures, to a chemical and/or biological outcome. This-process can be described as comprising the steps of:

I. identifying one or more subsets of molecules having given properties in relation to the chemical and/or biological outcome of interest;

II. generating a preliminary library comprising fragments of the molecules in said one or more subsets;

III. applying an algorithm to estimate the contribution of said fragments in relation to the chemical and/or biological outcome of interest; and

IV. obtaining a score value for each said fragment to which said algorithm is applied, which score values can be ranked in order of magnitude; whereby those fragments most likely to contribute to the chemical and/or biological outcome of interest, are associated with, e.g., high-ranking score values.

As mentioned above, the fragment library 120 contains the fragments as well as the obtained score values for the fragments. Once the fragment library 120 has been generated in step 230, the process may, or may not, perform a reiteration in step 240.

By embodying the DSA process in a reiterating manner, computational resources can be used in a very effective manner. For instance, the process preferably starts with small fragments. Since the number of possible fragments in molecular structures increases approximately exponentially with the maximum size of fragments that are investigated, this maximum size is set to a rather low value at the beginning so that even a very high number of molecular structures can be handled.

The process of steps 210 to 230 reveals fragments of high contribution to the desired activity. The revealed fragments can then be used in the next round (or cycle) to find fragments of greater size, i.e. higher molecular weight. An example of the reiteration process is depicted in FIG. 3. In the first round, the fragment C═O has been found as having a high contribution to the desired activity. This fragment is then used to search for fragments that are greater in size than the resulting fragment of the first round and that include this fragment. In the example of FIG. 3, the second round shows that the fragment N—C═O is the best fragment of this size with respect to the desired activity. This reiteration process is then continued, thereby increasing the size of fragments, and may lead to a compound that probably has the desired biological and/or chemical property and is suitable for the desired application.

Referring now back to FIG. 2, if it is decided in step 240 to perform a next round or cycle, the fragment library 120 generated in step 230 is analysed in step 250, and the process returns to step 220. Examples of how the fragment library 120 is analysed in step 250 will be described in more detail below. As will be appreciated, the reiteration process allows for applying more advanced functions such as generalisation functions 145 and annealing functions 155 to further improve the discovery process using discrete substructural analysis.

Finally, when it is decided in step 240 that no reiteration is to be performed, or the reiteration process has come to its end, the compound collection 125 is generated in step 260.

Turning now back to step 230 of generating the fragment library 120, a preferred embodiment of the substeps of this generation process will now be described with reference to FIGS. 4 to 6. First, after the internal database 115 and/or the external data source are accessed and a subset of molecules are identified, the structure activity data relating to the identified molecules is received in step 410. Then, fragments of the molecules in the subset are determined in step 420.

The molecules can be fragmented using a number of conventional techniques. For instance, an algorithm can be used for finding any permutation of atoms that are bonded with each other. The fragmentation functions 135 can employ a minimum size and a maximum size of fragments. To give another example, the fragmentation algorithm could be instructed to skip those fragments that have the atoms organised linearly. Further, the algorithm could be constrained to include or exclude certain types of bonds. There will be many different kinds of applying fragmentation functions that are easily available to the skilled practitioner.

That is, each of the molecular structures can conceptually be broken down into a series of discrete substructures or fragments (step 420). The fragments can be simple functional groups, e.g. NO₂, COOH, CHO, CONH₂; exact 2D substructures, e.g. o-nitrophenol; loosely defined families of substructures, e.g. R—OH; simple atoms or bonds, or any assemblage of structural descriptors in 2 or 3D chemical space.

After the molecules have been broken down to fragments in step 420, the fragment scores are computed in step 430 by calculating a score value for each fragment and associating the calculated value to the fragment. Then, the highest scoring fragments are determined in step 440 and stored in step 450.

An example of how the highest scoring fragments are determined is depicted in FIG. 5. In this example, the determined score values are plotted against the number of compounds that comprise the respective fragment. In this graph, each fragment is represented by a point. Using this plot in step 440 gives more information than just selecting the highest scoring fragments by comparing the score values, since the plot additionally uses the information on the number of compounds that include the respective fragments.

The process of finding the largest possible score value can be regarded as equivalent to generating a phylogenic mesh of hierarchically-related molecular fragments corresponding to a given biological and/or chemical activity. In this setting, the nodes of the mesh are supplied by the fragments themselves, and the likelihood that any single fragment is at the basis of the biological activity is given by the distance of the corresponding node from the origin, that is, the base of the mesh itself. Thus the larger the score value is for any given fragment, the farther the corresponding node is from the origin of the lattice and the more likely it is that the fragment represents a chemical solution to the, e.g., pharmacophore that is recognised by the target of interest.

The step 430 of scoring the fragments will now be described in more details with reference to FIG. 6. Applying scoring functions 140 corresponds to the aforementioned set of logical rules, or computational steps. The DSA method according to the invention comprises in a preferred embodiment the step of incorporating the variables relating to prevalence of each fragment into one or more mathematical functions that estimate the score value for any given fragment.

The said algorithm is a function of:

(a) the number of molecules x within a subset which meet a given threshold in relation to the desired outcome and which contain a given fragment;

(b) the number of molecules y within said subset which contain the said fragment, whether or not they meet said threshold;

(c) the number of molecules z within said subset which meet said threshold whether or not they contain the said fragment; and

(d) the number N of all molecules in the subset.

The outcome referred to in (a) may be any desired parameter relating to the activity of the compounds, including but not necessarily limited to biological, biochemical, pharmacological and/or toxicological activity. Each compound or molecule in the data set may then be analysed according to whether it possesses the desired parameter, in relation to a given threshold, such as a particular level of activity. The threshold can be set at any desired level. In the following description, an ‘active’ compound is one which meets the desired threshold and an ‘inactive’ compound is one which does not meet said threshold. The terms are not intended to express any absolute property of the compounds in question.

The contribution of a given fragment may be determined by applying to the variables x, y, z and N a measure of association or a score function 140. As is well known to those skilled in the art there are many possible measures of association, which fall into three main categories:

| | |
|---|---|
| Subtractive measures: e.g. | Nx − yz; |
| Ratio measures: e.g. | x(N − y − z − x)/(z − x)(y − x); |
| Mixed measures: e.g. | (x/z) − (z − x)/(N − z). |

It will be appreciated that any measure of association may be selected and those skilled in the art will readily be able to make the appropriate choice.

The algorithm applied in step 430 may therefore comprise (see FIG. 6):

(i) assessing the number of compounds x within a subset which meet a given threshold in relation to the chemical or biological outcome of interest and which contain a given chemical determinant (step 610);

(ii) assessing the number of compounds y within said subset of compounds which contain the said chemical determinant, whether or not they meet said threshold (step 620);

(iii) assessing the number of compounds z within said subset of compounds which meet said threshold whether or not they contain the said chemical determinant (step 630);

(iv) assessing the total number of compounds N within the subset of compounds (step 640); and

(v) applying a measure of association to two or more of the variables x, y, z and N (step 650), preferably three or four variables and most preferred all four variables x, y, z and N.

The measure of association may be applied directly, to determine a score value corresponding to the contribution of a given fragment. Preferably, however, the measure of association is developed into a score function, in order to assess the probability that a substructure contributes to an outcome. This facilitates a clearer determination of the ranking of the score values obtained for the totality of fragments analysed. The measure of association may be developed into a score function by methods well known in the art. For example the methods may conveniently be selected from statistical methods, e.g. critical ratio method (z); Fisher's Exact test, Pearson's chi-squared; Mantel Haenzel's chi-squared; and methods based on, but not limited to, performing inferences on slopes and the like. However, methods other than statistical tests may be used. Such methods include, but are not limited to the calculation and comparison of exact and approximate confidence intervals, correlation coefficients, or indeed any function containing measures of association comprised of a combination of one, two, three or four of the variables x, y, z or N described above.

Examples of mathematical formulae representing measures of association or score functions which may be employed in the present invention include:

x/z (I)

x/N (II)

Nx−yz (III)

(x/z)−(y/N) (IV)

(x/z)−(z−x)/(N−z) (V)

$$\begin{matrix}( VI ) \frac{x ( N - y - z + x )}{( z - x ) ( y - x )} & \\ ( VII ) \frac{Nx - yz}{\sqrt{z ( N - z ) y ( N - y )}} & \end{matrix}$$

e^{[(x/z)-(z-x)/(N-z)]} (VIII)

$$\begin{matrix}( IX ) \frac{{( \vert Nx - yz \vert - N / 2 )}^{2} N}{z ( N - z ) y ( N - y )} & \\ ( X ) \frac{x ( N - y - z + x )}{( z - x ) ( y - x )} {ⅇ}^{- 2 \sqrt{1 / x + 1 / ( y - x ) + 1 / ( z - x ) + 1 / ( N - y - z + x )}} & \\ ( XI ) \frac{{x}_{1} ( N - y - {z}_{1} + {x}_{1} ) ( {z}_{2} - {x}_{2} ) ( y - {x}_{2} )}{{x}_{2} ( N - y - {z}_{2} + {x}_{2} ) ( {z}_{1} - {x}_{1} ) ( y - {x}_{1} )} & \\ ( XII ) \frac{1}{\sqrt{d}} {\sum }_{i = 1}^{d} {( \sqrt{\frac{{( Nx - yz )}^{2} N}{z ( N - z ) y ( N - y )}} )}_{i} & \end{matrix}$$

The skilled practitioner in the field will recognize score function (VII) as a product moment correlation coefficient reflecting the degree of shared variance between two dichotomous variables not explicitely shown in said formula.

The skilled practitioner in the field will recognize score function (VIII) as being related to an estimation of a risk odds ratio using the slope of a regression line representing the degree of shared variance that exists between two dichotomous variables.

The skilled practitioner in the field will recognize score function (IX) as a chi-squared-related statistic modified for various confounding factors. For example, the term N/2 in the numerator of the second quotient of the product being logarithmically scaled is a conservative adjustment of the normal approximation to the binomial distribution, which is a useful modification for dealing with relatively small values of x, y, z or N. The skilled practitioner in the field will recognize that other measures of association and/or score functions can be used for the same purpose in lieu of those described in formulae (I) and (II), the most pertinent of which, in the sense of the present invention, contain various combinations of one, two, three or four of the variables x, y, z and N.

The skilled practitioner in the field will recognize score function (X) as a manner by which to estimate the value of the lower limit of the 95% confidence interval of measure (III), by using a logarithmic transformation to render the distribution of the ratio more comparable to that of the normal distribution, and a first order Taylor series approximation to estimate the variance of the logarithm of the same said ratio.

The skilled practitioner in the field will recognize score function (XI) as a way to compare odds ratios, allowing one to identify the chemical determinants that are most likely to be selective for one target over the other.

The skilled practitioner in the field will recognize score function (XII) as a way to combine multiple tests of association, allowing one to identify the chemical determinants that are most likely to have effects on two or more given properties at the same time.

The skilled practitioner in the field will also recognize that the score function may be modified to comprise additional variables related to a molecule's material, biological, chemical and/or physico-chemical properties. For example, such modifications could comprise, but in no way be limited to, adjustments for compound potency, selectivity, toxicity, bioavailability, stability (metabolic or chemical), synthetic feasibility, purity, commercial availability, availability of appropriate reagents for synthesis, cost, molecular weight, molar refractivity, molecular volume, logP (calculated or determined), number of H-bond accepting groups, number of H-bond donating groups, charges (partial and formal), protonation constants, number of molecules containing additional chemical keys or descriptors, number of rotatable bonds, flexibility indices, molecular shape indices, alignment similarities and/or overlap volumes.

Thus for example, score function (VIII) may be further modified e.g. to account for the molecular weight of each chemical determinant under consideration (MW) as follows:

MW·e^{[(x/z)-(z-x)/(N-z)]}

Similarly, score function (IX) may be modified to include the variables MW and [S], which respectively represent the molecular weight of a chemical determinant of interest (MW), and the number of times the same said chemical determinant appears in the subset of active compounds x ([S]), as follows:

$$( II ) Score = Log ( MW \cdot \frac{x}{[ S ]} \cdot \frac{{( \vert Nx - yz \vert - N / 2 )}^{2} N}{z ( N - z ) y ( N - y )} )$$

in order to favor the identification of the largest possible, singleton, biologically-active chemical determinants during the analysis.

The results of step 650 of the algorithm provides the score value of the fragment under consideration. Steps 610-650 of the algorithm may be repeated for each of the chosen fragments in the data. When the values for all the chosen fragments have been calculated, the results provide a score value corresponding to the potential efficacy of each of the fragments that have been analysed. Said score values can be ranked in order of magnitude; whereby those fragments most likely to contribute to the chemical and/or biological outcome of interest, are associated with, e.g., high-ranking score values. This enables in step 440 the identification of one or more local extrema of the values of the score function, whose corresponding chemical determinants represent full or partial chemical solutions to the desired chemical or biological outcome. Finding the largest score values that can be achieved in any given data set is equivalent to identifying the chemical determinants contained within subsets of molecules having the desired properties which chemical determinants have the lowest probability of occurring by chance in the same subsets. When the desired property is a given biological activity the highest scoring fragments or chemical determinants represent a biologically active pharmacophore.

Turning now back to FIG. 2, preferred embodiments of step 250 of analysing the fragment library 120 will now be discussed.

One way of analysing the fragment library 120 is depicted in FIG. 7. The process starts with selecting a fragment in step 710 based on the score values determined in the preceding round. Then, compounds from the previous set that contain the selected fragment are extracted in step 720. Since in step 710, a fragment of high contribution to the desired activity was selected, the compounds that are extracted in step 720 can be considered as active compounds. Then, in step 730, a set of inactive compounds is selected, either from the previous set or from the databases or any other source. Then, the active and inactive compounds are brought together in step 740 to form a new compound set. The new compound set is then selected in step 220 as the compound set of the next reiteration generation to proceed with the next round.

A preferred embodiment of performing step 730 will now be described with reference to FIG. 8. This embodiment makes use of generic substructures to select a new set of compounds for the next round.

The process of FIG. 8 starts with analyzing, in step 810, the structure of the fragment that was selected in step 710. When using the generic aspect of the invention, the fragment that is selected in step 710 can be selected by evaluating the score value that has been calculated in the previous round. Additionally, the fragment selection can be made dependent on further factors which influence the suitability of the fragment to be the starting point for the generalization. This suitability might be a function on the number of atoms or bonds, on the way of how the atoms are bonded, on the three-dimensional structure of the respective fragment, etc.

After the structure of the selected fragment has been analyzed in step 810, a generalized item is located in the fragment structure in step 820. This item is then replaced with a generalized expression in step 830 to result in a generic substructure (e.g. to find bio-isosters). An example is

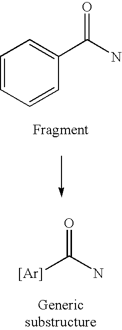

where in the given selected fragment, two generalized items have been located and replaced with the general expressions [Ar] and A, where [Ar] represents an aromatic center, and A represents C, or S.

The generic substructure generated in step 830 is then used to perform a virtual screening to find new compounds matching the generic substructure. The term “virtual screening” refers to any screening process that is performed with data only, thereby avoiding the need to synthesize compounds. The new compounds that are revealed by virtual screening, are then used to construct a new compound set in step 850 that can be used in the next reiteration round.

As can be seen from FIG. 9, the virtual screening process can be divided in intra- and extra- domain modifications of fragments brought on by the use of generic substructures. Intra-domain modifications performed in step 910 comprise substitutions, insertions, deletions and inversions of atoms of a fragment. Starting from the above-mentioned exact fragment and generalizing this fragment to the generic substructure, three different substitutions are obtained in the following example:

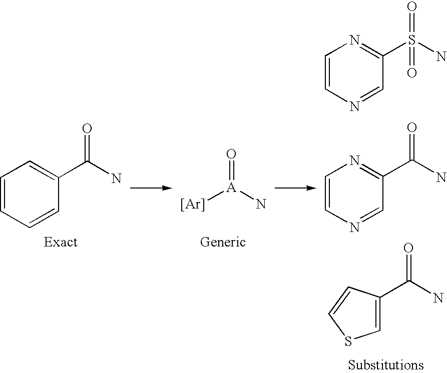

Extra-domain modifications performed in step 920 consist in changes in the substituants of a fragment. These can be random, focused, etc.:

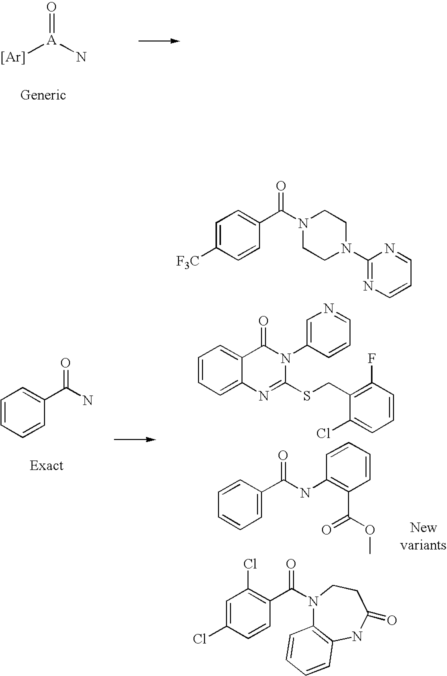

Focused compound sets are collections of molecules that are based on modifications of one or more generic substructures:

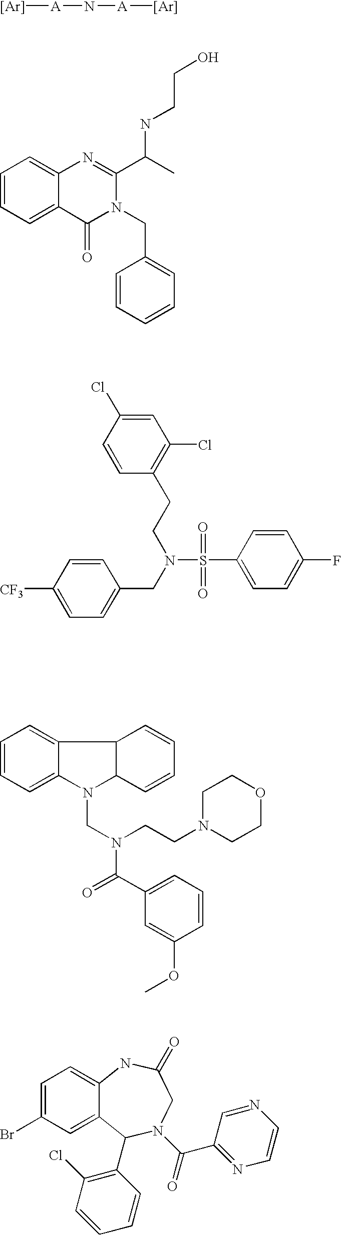

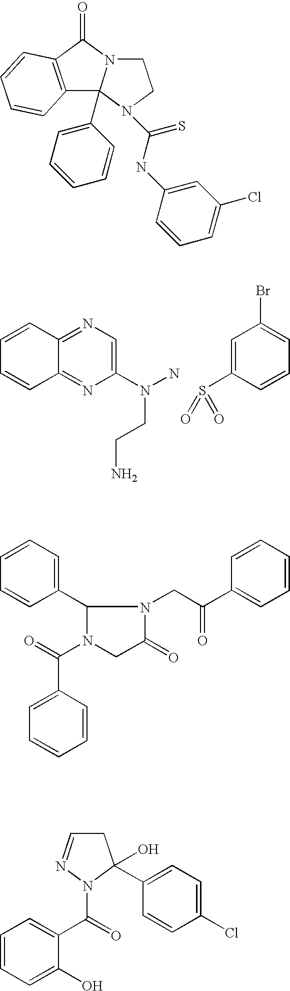

While in FIG. 9 the steps of performing intra- and extra-domain modifications are shown to be performed in series, it will be appreciated by those of ordinary skill in the art that it is within the invention to perform only one of these different kinds of modifications, or to perform both modifications in a different sequence or even in parallel. It is to be understood that the result of the virtual screening is a diverse collection of compounds that have a high chance of being active, as they are enriched with substructures associated with activity.

While in step 710, a fragment is selected that forms the basis for applying the generalization functions 145 to obtain a generic substructure, it is another preferred embodiment of the invention to select a greater number of high scoring fragments to generate generic substructures. For instance, the following fragments have been shown to have high contributions to the desired activity, and can be selected in step 710:

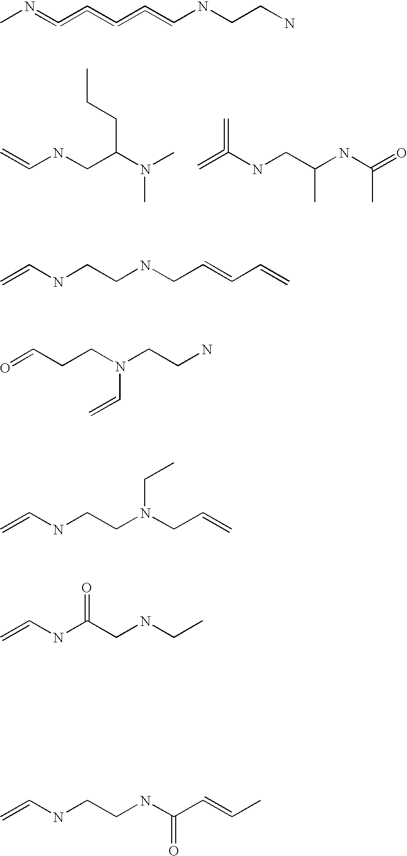

These selected fragments are then reduced to high scoring generic substructures such as:

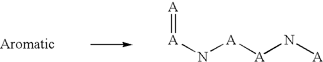

These generic substructures are then used to virtual screen commercial databases

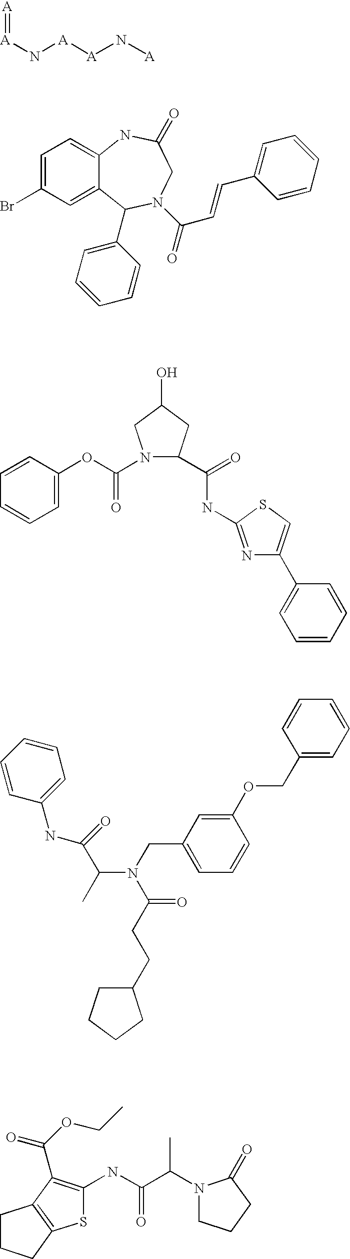

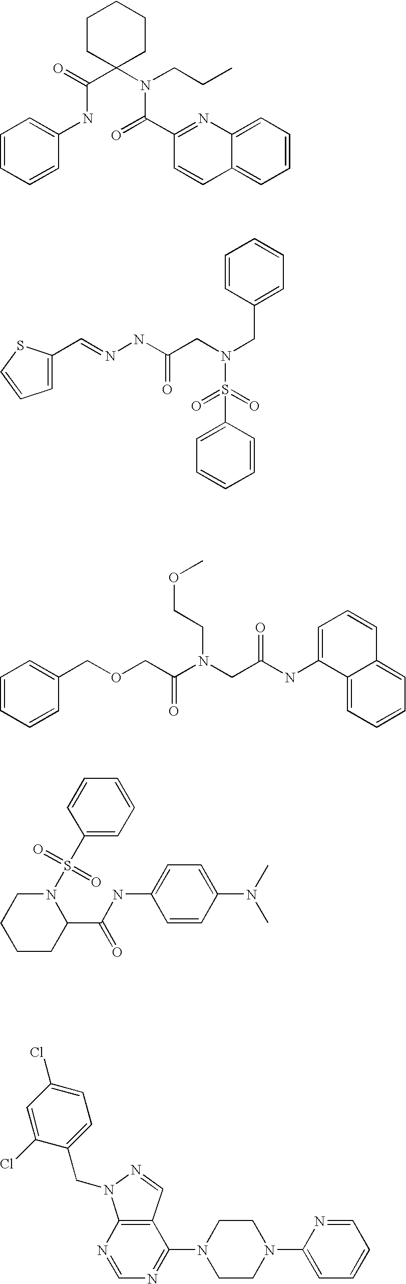

or corporate compound collections.

While the reiteration process has been described as being advantageous for computational reasons since it is useful to start with small fragments and increasing the fragment size from round to round, and while it has been further shown that the power of discovery can still be increased by using generic aspects in the reiteration process, there is yet another approach according to the invention of further improving the discrete substructural analysis process of the invention. This further approach is based on an annealing technique and will now be described with reference to FIG. 10.

In the preferred embodiment of FIG. 10, the step 250 of analyzing the fragment library that has been generated in the previous round, starts with steps 1010 and 1020 of selecting a first and a second fragment. Both fragments are selected based on the calculated score values and can be understood as being high contributing fragments.

In the following step 1030, an annealing function 155 is applied for connecting the first and the second fragments. Connecting the fragments means to define a molecular structure or substructure including both fragments. For this purpose, a number of different annealing functions 155 can be used. These annealing functions differ in the concrete implementation of how certain annealing parameters are evaluated and used. Annealing parameters are, e.g., the (predetermined) distance of the first and second fragments, the three-dimensional orientation of the first and the second fragments, the number of atoms that are put between the fragments, the number of bonds that are used for gluing the fragments together, the kind of bonds and atoms, etc.

Further, the annealing process is preferably combined with the generic aspect described above. If for example in steps 1010 and 1020 fragments F1 and F2 are selected that are known to have high score values, the annealing function that is selected in step 1030 and run in step 1040 might use the generic expression

F1—[G]—F2

to connect the fragments. The general expression [G] is a synonym for molecular substructures of given properties and annealing parameters and depends on the annealing function used.

Once the fragments have been combined, by means of exact or generic expressions, a new compound set is generated in step 1040 that includes both fragments. An example of a molecule of the new compound set is depicted in FIG. 11 which is a two-dimensional relative contribution map showing the relative contribution in relation to the local coordinates. As can be seen from FIG. 11, there are two local maxima showing the approximate score values of 1.2 and 1.7 of the fragments F1 and F2.

The annealing process is advantageous for two reasons. The first advantage is that by connecting two fragments having high contribution to the desired activity, larger molecules can be obtained that participate from the fact that they include more than one high scoring fragment. The resulting structures have therefore good chances to have an even higher score value than the highest score value of the two fragments.

For instance, in the structure of FIG. 11, the resulting compound includes fragments having score values of 1.2 and 1.7 but may result in a total score value for the entire structure of, e.g., 2.1. The annealing technique therefore allows for discovering compounds of even higher activity.

The second advantage is that the annealing technique allows to avoid deadlocks in the computational process. As can be seen from FIG. 11, the relative contribution values indicate two local maxima. When performing the reiteration process depicted in FIG. 3, starting with small fragments and increasing the fragment size in each reiteration from round to round, a deadlock may arise when the selected fragment in one of the intermediate steps is located on a local maximum.

For instance, when at the end of the second round the fragment N—C═O is selected and this fragment is located on a local maximum, the next round will not be successful. As described above, the fragments of the next round are preferably constructed from the selected fragment of the previous round by incrementally increasing the fragment size. Thus, whatever atom is added to the selected fragment, the next round will shift the fragment away from the local maximum. That is, any resulting fragment has a lower score value than the selected fragment of the previous round in this case.

To avoid this deadlock, the annealing technique can be applied by selecting two good fragments from the previous round, connecting the fragments, calculating a score value and continuing the process. This can be done periodically from round to round, or whenever a deadlock is detected.

While the invention has been described using a number of preferred embodiments, it will appreciated by those of ordinary skill in the art that the invention is by no means limited to these embodiments. For instance, the sequence of method steps shown in the flowcharts can be changed, or steps that are shown to be performed consecutively could be even performed in parallel, see e.g. steps 1010 and 1020 of the process shown in FIG. 10.

Further, it is apparent to those of ordinary skill in the art that not all of the shown method steps are required in any implementation. For instance, in the scoring process of FIG. 6, parameters that are not used by the scoring function are not required to be calculated. Further, the parameters could be calculated in parallel using a multi-tasking or multi-threading operating system.

Further embodiments of the invention will now exemplarily be described.

For instance, the library of fragments generated in step 230 may in theory contain all possible fragments and combinations thereof. This may be achieved in practice if the library is generated by computer. However, if the library is generated manually, it is likely to contain only a selection of all possible fragments. The method may therefore be repeated using combinations of fragments, in particular combinations of fragments for which high score values have been obtained in a previous analysis.

Thus, following an initial analysis of fragments, those fragments most likely to contribute to the chemical and/or biological outcome of interest may be combined and an algorithm applied as described hereinbefore to estimate the contribution of said combined fragment in relation to the chemical and/or biological outcome of interest. The score value obtained can be compared with the score values of the individual fragments to verify whether the combination results in an improvement of the contribution to the chemical and/or biological outcome of interest.

In a further embodiment of the present invention, it may be possible to single out from the fragments having the greatest contribution to the chemical and/or biological outcome of interest a common structural portion to identify if the contribution of said common portion is the same or higher than the starting fragments.

The fragments with the highest score values, represent the chemical determinant or molecular fingerprint having the largest weighting for contribution to a given chemical or biological outcome

Having identified said fingerprint it is then possible to create a library of compounds containing said chemical determinant(s). The compounds may be obtained by a program of synthesis, around the structural feature in question. Alternatively, compounds containing the chemical determinant may be identified from commercial catalogues and purchased from the relevant source. The compounds will not necessarily have been prepared for pharmaceutical purposes and may be available from a variety of sources.

Once the desired library has been assembled, it can be screened against the target(s) of interest. The results of the screening may identify compounds which are sufficiently active to develop further, or may provide leads for a program of synthesis. The DSA method according to the present invention enables the creation of diverse, yet highly focused libraries, in relation to a particular biological or pharmacological target. Thus the likelihood of success in screening for active compounds and/or useful leads is much increased.

In a further embodiment, the present invention provides a method for the identification of molecules having certain desired properties, such as biologically active molecules, which method comprises:

Weighting the contribution of molecular fragments, within a subset of molecules, to a given chemical or biological outcome as described hereinabove,

Identifying one or more fragments with the highest weighting, and

Compiling a set of compounds which compounds contain one of more of said fragments and optionally

Testing said compounds for the desired properties.

It will be appreciated that the method may equally be used to identify fragments which lead to undesired properties, e.g. adverse biological side effects and hence to exclude from consideration compounds having said fragments.

Thus the process of the present invention generates structural hypotheses (fragments) whose likelihood of being an explanation to a given biological, biochemical, pharmacological or toxicological outcome is estimated by calculating a quantitative score value. Considering the score value for a given fragment enables the drug developer to make informed decisions as to the approach which is most likely to achieve a desired goal, such as the identification of more potent compounds, the discovery of new series of active compounds, the identification of more selective or more bio-available compounds or the elimination of toxic effects.

The method of the present invention focuses on the fragments present within the subset of compounds of interest, thereby eliminating the need to perform tedious calculations for vast, but more likely less relevant sectors of chemical space. This results in a reduction in the number of computational steps that are needed to address a given biological outcome, whilst retaining the basic level of molecular understanding that is required in order to postulate the existence of biologically active chemical determinants.

As discussed hereinbefore, the process of the invention involves searching for local extrema of one or more functions, which can be readily selected so that these correspond to probabilities given in common statistical tables. This provides an elegant method of evaluating the potential contribution of a given fragment to a chemical or biological outcome. However, it is not necessary to base the analysis on statistical theory in order to carry out the invention.

The DSA method of the invention can be used in a wide range of drug discovery applications. As described hereinabove the method enables the identification of pharmacophores which have a high probability of contributing to a given biological activity, for example, 7-TM receptor antagonists, kinase inhibitors, phosphatase inhibitors, ion channel blockers, and protease inhibitors as well as the active moieties of naturally occurring peptidergic ligands.

The method also allows the identification of endogenous modulators of drug targets, facilitating the identification of new axes of pharmacological intervention, as well as the rational incorporation of novel pharmacological properties into molecules previously devoid of such said properties.

The method may also be utilised to identify false positive and false negative results in data sets, for example those derived from high throughput screening. DSA is also of use in predicting compound selectivity, for example by identifying potentially undesirable secondary effects.

The method can be used in the same way to predict the toxic effects of a compound, by identification of its “toxicophoric” chemical determinants, which in conjunction with the above, allows for the construction of chemical determinant databases of great use for chemical series selection. In this context, the method further allows for the rational incorporation of novel pharmacological properties into chemical compounds previously devoid of such activities. Finally, and via its capacity to identify the most appropriate level of molecular diversity that needs to be tested during a screening campaign the DSA method allows for the efficient conduct of rational, massively parallel, automated high-throughput screening campaigns, which is a marked improvement over the current HTP discovery strategies.

It will be appreciated that in the above method, at least one step is effected by a computer-controlled system. Thus, for example, the values x, y, z and N obtained from the database(s) may be entered into and processed by a suitably programmed computer. The present invention therefore extends to such computer-controlled or computer-implemented methods.

From the above description, it is apparent that the present invention provides a new method for the rapid identification of molecules having certain desired properties such as biologically active molecules. In particular the invention relates to a method of weighting the efficacy of molecular structures, in order to identify the biologically active moieties of molecular structures, and using these moieties in the design of focused chemical compound collections for more rapid and cost-effective drug discovery.

A method is provided for increasing the proportion of biologically active compounds in a given set of chemical entities wherein said entities are not already known to have the desired biological activity. The said method involves the application of various mathematical techniques to the determination of quantitative structure activity relationships (QSAR). This new method, which may be termed discrete substructural analysis (DSA) provides a solution e.g. to the problem of pharmacological pattern recognition, that is, the problem of identifying the chemical determinants (CD's) that are responsible, with regard to a given compound, for any given chemical or biological outcome, which may be for example the biological, biochemical, pharmacological, chemical and/or toxicological activity.

The method of the present invention has wide application and is not restricted to the pharmaceutical field. In terms of biologically active compounds the method may for example be used in connection with pesticides and herbicides, where the desired biological activity is respectively pesticidal and herbicidal activity. The method may also be used in reactive modelling applications where the desired properties are chemical rather than biological attributes, e.g. in the preparation of catalysts.

It will be appreciated that it is a technique of the invention to combine within a subset or between different subsets those fragments most likely to contribute to the chemical and/or biological outcome of interest, and apply an algorithm to estimate the contribution of said combined fragment in relation to the chemical and/or biological outcome of interest, whereby the score value obtained can be compared with the score values of the individual fragments to verify whether the combination results in an improvement of the contribution to the chemical and/or biological outcome of interest.

Further, the invention allows to single out from the fragments having the greatest contribution to the chemical and/or biological outcome of interest a common structural portion to identify whether the contribution of said common portion is the same or higher than the starting fragments.

Moreover, a measure of association is used that is preferably selected from subtractive measures, ratio measures or mixed measures. The measure of association is preferably incorporated in, or developed into, a score function. The score function can be developed using a statistical method selected from the critical ratio method, Fisher's Exact test, Pearson's chi-squared, Mantel Haenzel's chi-squared, inference on slopes and the like. It is another preferred embodiment that the score function is developed using a method selected from the calculation and comparison of exact and approximate confidence intervals, correlation coefficients or any function explicitly containing a measure of association comprising any combination of one, two, three or four of the variables x, y, z and N.

Preferably, the invention performs the step of selecting molecules containing the highest-ranking fragments as potential ligands and optionally testing them subsequently as modulators of a drug target. The process of the invention can preferably be used to identify false positive and/or false negative experimental results. Other preferred applications are to perform similarity searches, diversity analysis and/or conformation analysis.

In the following, examples are given showing the numerous applications of the DSA process according to the invention. The examples are preferred embodiments of the invention and serve to illustrate the invention, but are not to be considered as limiting its scope.

### EXAMPLE NO. 1

### Rational Identification of Novel and Selective Receptor Ligands

A competition binding assay was developed for a cell surface receptor using a recombinant membrane preparation and a radiolabelled peptide. A collection of compounds for testing in the assay was assembled, tested, and novel receptor ligands were identified according to the method of the present invention. The first step consisted in the compilation of a list of 208 structures of antagonists of the same said receptor by reviewing the current scientific literature. The second step consisted in identifying the biologically-active chemical determinants contained within these 208 receptor ligands. For this means, an additional list containing 101′130 structures described as having no effect on the same said receptor was generated, and added to the first. The resulting list of 101′338 structures was then analyzed for the presence of biologically-active chemical determinants by selecting a subtractive measure of association (I), wherein x represented the number of active chemical structures containing a chemical determinant of interest, y represented the total number of chemical structures containing the same said chemical determinant, z represented the total number of active chemical structures in the set of N molecules (i.e. z=208), and N represented the total number of chemical structures subject to analysis (i.e. N=101′338).

Nx—yz (I)

Measure of association (I) was then developed into score function (II), which the skilled practitioner in the field will recognize as an indirect measure of the probability of chance occurrence modified for various confounding factors. For example, the term N/2 in the numerator of the second quotient of the product being logarithmically scaled is a conservative adjustment of the normal approximation to the binomial distribution, which is a useful modification for dealing with relatively small values of x, y, z or N. The variables MW and [S], which respectively represent the molecular weight of a chemical determinant of interest (MW), and the number of times the same said chemical determinant appears in the subset of active compounds x ([S]), were included in the score function in order to favor the identification of the largest possible, singleton, biologically-active chemical determinants during the analysis. The skilled practitioner in the field will recognize that other measures of association and/or score functions can be used for the same purpose in lieu of those described in formulae (I) and (II), the most pertinent of which, in the sense of the present invention, contain various combinations of two, three or four of the variables x, y, z and N.

$$( II ) Score = Log ( MW \cdot \frac{x}{[ S ]} \cdot \frac{{( \vert Nx - yz \vert - N / 2 )}^{2} N}{z ( N - z ) y ( N - y )} )$$

The skilled practitioner in the field will also recognize that score function (II) could also be modified to comprise additional variables related to a molecule's material, biological, chemical and/or physico-chemical properties. For example, such modifications could comprise, but in no way be limited to, adjustments for compound potency, selectivity, toxicity, bioavailability, stability (metabolic or chemical), synthetic feasibility, purity, commercial availability, availability of reagents for synthesis, cost, molecular weight, molar refractivity, molecular volume, logP (calculated or determined), prevalence of a given substructure in a collection of drug-like molecules, total number and/or types of atoms, total number and/or types of chemical bonds and/or orbitals, number of H-bond accepting groups, number of H-bond donating groups, charges (partial and formal), protonation constants, number of molecules containing additional chemical keys or descriptors, number of rotatable bonds, flexibility indices, molecular shape indices, alignment similarities and/or overlap volumes.

Analysis of the 101′338 structures led to the identification of eight distinct chemical determinants, ranging from 150 to 230 Da in molecular weight, and having less than a 1 in 10′000 probability of being contained within the subset of active chemical structures on the basis of chance alone (p<0.0001). Accordingly, the eight chemical determinants were accepted as being representative of one or more biologically active moieties of the 208 receptor ligands derived from the literature, and were assembled into a fourth list. Calculations using formula (II) were then reiterated in order to ascertain if a larger chemical determinant resulting from the combination or further expansion of any of the eight fragments could be identified. The largest, statistically significant, chemical determinant found in these additional calculations had a molecular weight of 335 Da, and was selected as a representative scaffold, or pharmacologically active “fingerprint” for subsequent compound selection and synthesis. The third step of the process involved using the representative scaffold described above as a template for virtual screening and compound selection. For this means, substructure searches were conducted in a database of over 600′000 commercially available compounds, using both the calculated fingerprint and fragments thereof. A total of 1360 compounds were acquired on the basis of these searches, and an additional 1280 compounds were randomly selected and acquired from the same suppliers for control purposes.

The fourth and fifth steps, constituting the final phases of the process, were conducted in parallel. The fourth step involved testing the two sets of compounds described above in the radioligand binding assay. Of the 1360 molecules selected on the basis of the representative scaffold, 205 molecules showed competitive activity when assayed at concentrations ranging between 1 and 10 μM, 21 compounds showed activity when tested at concentrations ranging between 0.1 and 1 μM, and one compound, termed compound A, displayed an affinity for the receptor (Ki) of 8.1±1.05 nM (n=12). Each of the 1280 randomly selected compounds failed to demonstrate receptor binding properties when tested at a concentration of 10 μM. As such, the set of compounds compiled on the basis of a representative fingerprint was at least 21-fold more effective in delivering active molecules than was the set of random compounds (p<0.0001).

Compound A was found to represent a novel, hitherto unreported, class of inhibitor of the receptor of interest. FIG. 12 illustrates the effect of compound A on receptor-mediated inositol trisphosphate generation. Cells expressing the receptor of interest were preloaded with radiolabelled inositol, and exposed to receptor agonist in the presence of increasing concentrations of compound A. Inositol trisphosphate (IP₃) generation was measured following elution of radiolabelled cellular inositol phosphates from an affinity column. Compound A inhibited agonist-induced IP₃ generation with an IC₅₀ of 22 nM, a value consistent with the affinity of the compound for the receptor.

As shown in FIG. 12, compound A significantly reduced receptor-mediated inositol trisphosphate generation in a cell-based functional assay (IC₅₀=22 nM), a finding consistent with both the compound's affinity for the receptor, and the use of receptor antagonists in the calculations described above. Finally, compound A was determined as being highly selective for the receptor of interest, insofar as it failed to demonstrate significant inhibitory activity when tested at a concentration of 10 μM in more than 20 other radioligand receptor binding assays.

The fifth step consisted in using the representative scaffold described above to direct the conceptual design and synthesis of novel chemical compounds, in the sense of composition of matter, and in view of identifying novel molecules with receptor-binding activities. For this means, a list of chemical reactants and reaction products was assembled, wherein the biologically active representative scaffold described above, or fragments thereof, were contained either within the chemical structures of the reactants, or within the resulting reaction product(s). More than 2000 combinations of reactants were selected, and the corresponding reaction products were synthesized for testing. Testing these compounds in the receptor binding assay led to the identification of a novel class of chemical compound in the sense of composition of matter, a number of representatives of which displayed IC₅₀s in the 50 to 500 nM range.

### EXAMPLE NO. 2

### Rational Identification of Novel and Selective Kinase Inhibitors

An enzymatic assay was developed for a human kinase involved in inflammation, for which no inhibitors were previously described in the literature. A collection of compounds for testing in the assay was assembled, tested, and novel kinase inhibitors were identified according to the method of the present invention. The first step consisted in the compilation of a list of 2367 chemical structures of inhibitors of purine nucleotide-binding proteins from the scientific literature, including the structures of compounds shown to inhibit other kinases, phosphodiesterases, purine nucleotide-binding receptors, and purine nucleotide-modulated ion channels, henceforth referred to as “surrogate targets”. The second step consisted in identifying the biologically-active chemical determinants contained within these 2367 chemical structures. For this means, an additional list containing 98′971 structures described as having no effect on the same said surrogate targets was generated, and added to the first. The resulting list of 101′338 structures was analyzed for the presence of biologically-active chemical determinants by selecting a ratio measure of association (III), wherein x represented the number of active chemical structures containing a chemical determinant of interest, y represented the total number of chemical structures containing the same said chemical determinant, z represented the total number of active chemical structures in the set of N molecules (i.e. z=2367), and N represented the total number of chemical structures subject to analysis (i.e. N=101′338).

$$( III ) \frac{x ( N - y - z + x )}{( z - x ) ( y - x )}$$

Measure of association (III) was then developed into score function (IV), which the skilled practitioner in the field will recognize as a manner by which to estimate the value of the lower limit of the 95% confidence interval of measure (III), by using a logarithmic transformation to render the distribution of the ratio more comparable to that of the normal distribution, and a first order Taylor series approximation to estimate the variance of the logarithm of the same said ratio. In this instance, no additional variables other than x, y, z or N were used in the score function, although it is apparent to the skilled practitioner in the field that formula (IV) could also be modified to comprise additional variables related to a molecule's material, biological, chemical and/or physico-chemical properties, as mentioned, but not limited to, those cited in example No. 1. The skilled practitioner in the field will also recognize that other measures of association and/or score functions can be used for the same purpose in lieu of those described in formulae (III) and (IV), the most pertinent of which, in the sense of the present invention, contain various combinations of two, three or four of the variables x, y, z and N.

$$( IV ) Score = \frac{x ( N - y - z + x )}{( z - x ) ( y - x )} {ⅇ}^{- 2 \sqrt{1 / x + 1 / ( y - x ) + 1 / ( z - x ) + 1 / ( N - y - z + x )}}$$

The analysis of the 101′338 chemical structures annotated for various biological activities was conducted by scoring a series of chemical determinants with formula (IV), until one or more groups of determinants were recognized as containing elements having a value greater than one, which corresponded to a less than 1 in 20 probability of being contained within the subset of biologically active structures on the basis of chance alone (p<0.05). Accordingly, these chemical determinants were accepted as being representative of one or more pharmacologically active moieties of inhibitors of the surrogate targets described in the literature, and were assembled into a fourth list. As opposed to searching for maximally scoring combinations of these determinants as described in example No.1, the structures were directly used as representative scaffolds, or pharmacologically active “fingerprints” for subsequent compound selection and synthesis.

The third step involved using the representative scaffolds described above as templates for virtual screening and compound selection. For this means, substructure searches were conducted in a database of over 250′000 commercially available compounds, using both the calculated fingerprints, fragments, and combinations thereof. A total of 2846 compounds were acquired on the basis of these searches, and the same collection of 1280 randomly selected compounds described in example No. 1 was used for control purposes.

The fourth and fifth steps, constituting the final phases of the process, were conducted in parallel. The fourth step involved testing of the acquired compounds in the enzyme assay. Of the 2846 molecules selected on the basis of representative scaffolds, 88 molecules showed inhibitory activity when tested at a concentration of 5 μM. Among these, six molecules displayed IC₅₀s in the 0.2 to 2 μM range, and one compound, termed compound B, displayed an IC₅₀ of 164 nM (FIG. 13).

FIG. 13 illustrates the effect of compound B on kinase-dependent protein phosphorylation. The kinase of interest was incubated with radiolabelled ATP and peptide substrate in the presence of increasing concentrations of compound B. Protein phosphorylation was measured using standard radiometric techniques. Compound B significantly inhibited kinase-dependent phosphorylation of protein substrate, displaying an IC₅₀ of 164 nM.

Among the 1280 randomly selected compounds tested for control purposes, only three showed inhibitory activity in the screening assay, the most potent of which displayed an IC₅₀ of only 7.8 μM. As such, the set of compounds compiled on the basis of representative fingerprints was 13.2 fold more effective in delivering active molecules than was the set of randomly selected compounds (p<0.0001). Furthermore, compound B was found to represent a novel, hitherto unreported, class of ATP-competitive kinase inhibitor, showing greater than 250-fold selectivity for the kinase of interest when tested in selectivity assays using both structurally- and functionally-related, alternative kinases.

The fifth step consisted in using one or more of the representative scaffold(s) described above to direct the conceptual design and synthesis of novel chemical compounds, in the sense of composition of matter, and in view of identifying novel molecules with kinase-inhibitory activities. For this means, a list of chemical reactants and reaction products was assembled, wherein the biologically active representative scaffolds described above, or fragments thereof, were contained either within the chemical structures of the reactants, or within the resulting reaction product(s). More than 4000 combinations of reactants were selected, and the corresponding reaction products were synthesized for testing. Testing these compounds in the screening assay led to the identification of two novel classes of chemical compounds, in the sense of composition of matter, a number of representatives of which displayed IC₅₀s in the 100 to 500 nM range.

### EXAMPLE NO. 3

### Rational Identification of Novel and Selective Ion Channel Blockers

An assay was developed for an ion channel believed to play a role in neurodegeneration, for which no inhibitors were previously described in the literature. A collection of compounds for testing in the assay was assembled, tested, and novel inhibitors were identified according to the method of the present invention. The first step consisted in generating the necessary structural data for identifying the chemical determinants of inhibitors of the channel of interest. This was accomplished by testing the first 3680 compounds of our corporate collection at a 5 μM concentration in the screening assay, and annotating each structure in the list for its inhibitory activity. Using a cutoff of 40% inhibition as a threshold for classification, 36 structures were identified as being active, and the remaining 3644 compounds were qualified as inactive.

The second step consisted in identifying the biologically active chemical determinants contained within the structures of the 36 inhibitors. For this means, the 3680 annotated structures were analyzed by selecting the previously described measure of association (I), wherein x represented the number of active chemical structures containing a chemical determinant of interest, y represented the total number of chemical structures containing the same said chemical determinant, z represented the total number of active chemical structures in the set of N molecules (i.e. z=36), and N represented the total number of chemical structures subject to analysis (i.e. N=3680). Measure of association (I) was then developed into score function (V), which the skilled practitioner in the field will recognize as a product moment correlation coefficient reflecting the degree of shared variance between two dichotomous variables not explicitely shown in formula (V).

$$( V ) Score = \frac{Nx - yz}{\sqrt{x ( N - z ) y ( N - y )}}$$

In this instance, no additional variables other than x, y, z or N were used in the score function, although it is apparent to the skilled practitioner in the field that score function (V) could also be modified to comprise additional variables related to a molecule's material, biological, chemical and/or physico-chemical properties, as mentioned, but not limited to, those cited in example No. 1. The skilled practitioner in the field will also recognize that other measures of association and/or score functions can be used for the same purpose in lieu of those described in formulae (I) and (V), particularly as score function (V) is not invariant over different changes in study design and/or distributions of y, (N−y), z and (N−z). The most pertinent of these alternative methods, in the sense of the present invention, contain various combinations of two, three or four of the variables x, y, z and N.

The following panels show examples of chemical determinants used for analysis and selected for follow-up. A total of 3680 structures annotated for channel inhibiting activity were tested for the presence of biologically active substructures using a set of chemical determinants comprising the five illustrated in panel A. Among the five structures, determinant No. 4 displayed the highest score value, indicating that it had the highest likelihood of being at the basis of channel inhibiting activity. Accordingly, calculations were reiterated for structures containing determinant No. 4, and the chemical structure shown in panel B was identified as being one of the largest, statistically significant determinants contained within the set of 36 inhibitors, and was subsequently selected for follow-up. Symbols: A represents C, N, O, or S; B represents H or OH.

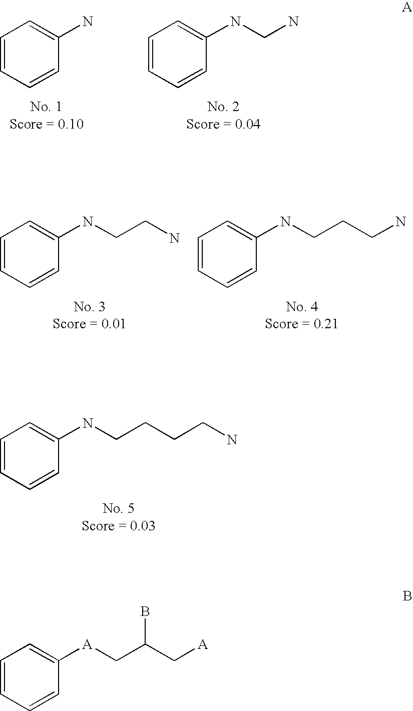

Analysis of the 3680 annotated structures was conducted by scoring a series of chemical determinants with formula (V), and by retaining the structures yielding the largest, non-null positive values. Examples of some of the chemical determinants used in this process are shown in panel A, along with their calculated score values. Among these, determinant No. 4 showed the highest score, and was estimated as having less than a 1 in 100 probability of being contained within the subset of channel blocking structures on the basis of chance alone (p<0.01). Accordingly, determinant No. 4 was accepted as being representative of a biologically active moiety of a large proportion of the 36 inhibitors, and calculations using formula (V) were then reiterated in order to ascertain if even larger chemical determinants could be identified. The largest, statistically significant, chemical determinant found in these additional calculations is shown in panel B. The structure was selected as a representative scaffold, or pharmacologically active “fingerprint” for subsequent compound selection and synthesis.

The third step involved using the representative scaffold described in panel B as a template for virtual screening and compound selection. For this means, substructure searches were conducted in a database of over 400′000 commercially available compounds, using both the calculated fingerprint and fragments thereof for this purpose. A total of 1760 compounds were acquired on the basis of these searches, and the same collection of 1280 randomly selected compounds described in example No. 1 was used for control purposes.

The fourth and fifth steps, constituting the final phases of the process, were conducted in parallel. The fourth step involved testing of the acquired compounds in the enzyme assay. Of the 1760 molecules selected on the basis of representative scaffolds, 84 molecules showed inhibitory activities of at least 40% when tested in the assay at a concentration of 5 μM. Among these, 8 molecules displayed IC₅₀s in the submicomolar range, and one compound, termed compound C, displayed an IC₅₀ of 400 nM. Two examples of these channel-inhibiting compounds are shown below, both of which contain the exact pharmacologically active “fingerprint” shown in panel B:

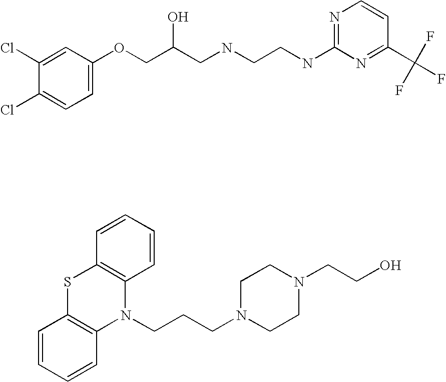

These two channel-inhibiting compounds were selected for testing using the method of the present invention. Both molecules significantly inhibited the channel of interest. As shown by the substructures highlighted in black, the chemical structures of the two compounds contain the pharmacologically active chemical determinant identified using the method of the present invention, and shown in panel B above.

Among the 1280 randomly selected compounds tested for control purposes, a total of 33 molecules showed an inhibitory activity of at least 40% in the screening assay. As such, the set of compounds compiled on the basis of the representative fingerprint shown in panel B, was 1.8 fold more effective in delivering active molecules than was the set of randomly selected compounds (p<0.005). The set of compounds compiled on the basis of the representative fingerprint shown in panel B was also 4.9 fold more effective in delivering active molecules than were the first 3680 compounds of the corporate compound collection (p<0.0001).

The fifth step consisted in using the representative scaffold shown in panel B, to direct the conceptual design and synthesis of novel chemical compounds, in the sense of composition of matter, and in view of identifying novel molecules with channel inhibiting properties. For this means, one of the 120 pharmacologically active inhibitors described above was selected for follow-up, and chemically modified using the previously assembled positive and negative screening results as a source of structure-activity information. This work led to the synthesis and subsequent identification of a novel, hitherto undescribed class of ion channel blocker, in the sense of composition of matter, a number of representatives of which displayed IC₅₀s in the 100 to 500 nM range. Selectivity testing indicated that the compound was selective for the channel of interest over 30 other drug targets, and further inhibited cell death in a model of nerve growth factor withdrawal-induced apoptosis.

### EXAMPLE NO. 4

### Rational Identification of Novel and Selective Protease Inhibitors

An enzyme assay was developed for a protease believed to play a key role in ischemic damage and injury. The protease in question was a member of a family of closely-related enzymes, itself being the only target of interest for therapeutic intervention. A collection of compounds for testing in the assay was assembled, tested, and novel enzyme inhibitors were identified according to the method of the present invention. The first step consisted in generating the necessary structural data for identifying the chemical determinants of inhibitors of the enzyme. This was accomplished by testing a collection of 1680 compounds at a 3 μM concentration in the screening assay, and annotating each structure for inhibitory activity. Using a cutoff of 40% inhibition as a threshold for compound classification, 17 structures were identified as being active, and the remaining 1663 molecules were qualified as inactive.

The second step consisted in identifying the biologically active chemical determinants contained within the structures of the 17 inhibitors. For this means, the 1680 annotated structures were analyzed by selecting the mixed measure of association shown below (VI), wherein x represented the number of active chemical structures containing a chemical determinant of interest, y represented the total number of chemical structures containing the same said chemical determinant, z represented the total number of active chemical structures in the set of N molecules (i.e. z=17), and N represented the total number of chemical structures subject to analysis (i.e. N=1680). In this instance, measure of association (VI) was directly used as a score function for identifying the biologically active chemical determinants contained within the 17 inhibitors of interest.

$$( VI ) \frac{x}{z} - \frac{y}{N}$$

In this context, no additional variables other than x, y, z or N were used in the score function, although it is apparent to the skilled practitioner in the field that formula (VI) could also be modified to comprise additional variables related to a molecule's material, biological, chemical and/or physico-chemical properties, as mentioned, but not limited to, those cited in example No. 1.

The skilled practitioner in the field will also recognize that other measures of association and/or score functions can be used for the same purpose in lieu of those described in formula (VI), particularly as the direct use this measure of association only allows for a relative estimation of the likelihood that a given chemical determinant is at the basis of biological activity. The most pertinent of these alternative methods, in the sense of the present invention, contain various combinations of two, three or four of the variables x, y, z and N.

Analysis of the 1680 annotated structures was conducted by scoring a series of chemical determinants with formula (VI), and retaining structures yielding the largest positive values. Examples of some of the chemical determinants used in this process are shown below in panel A, along with their calculated score values. Among these, determinants No. 7 and 8 showed the highest scores, and were accepted as being representative of one or more biologically active moieties contained within a substantial proportion of the 17 inhibitors. Calculations using formula (VI) were then reiterated in order to ascertain if an even larger chemical determinant could be identified, which was not the case using the available collection of 17 structures, and determinants No. 7 and 8 were merged together to form the representative scaffold, or pharmacologically active “fingerprint” shown below in panel B, which was subsequently used for compound selection and synthesis.

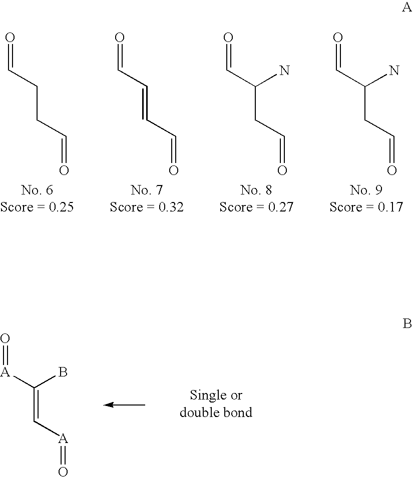

In the panels, examples are shown of chemical determinants used for analysis and selected for follow-up. A total of 1680 structures annotated for protease inhibiting activity were tested for the presence of biologically active substructures using a set of chemical determinants comprising the four illustrated in panel A. Among the four structures, determinants No. 7 and 8 displayed the highest score values, indicating that they had the highest likelihood of being at the basis of protease inhibiting activity. The determinant consisting of a simple benzene ring scored 0.02 in comparison. As no higher scoring structures were identified when reiterating calculations with determinants No. 7 and 8, the two structures were merged into the chemical motif shown in panel B, which was subsequently used as a pharmacologically active “fingerprint” for virtual screening and compound selection. Symbols: A represents C or S; B represents H, C, N, O, or any halogen atom.

The third step involved using the representative scaffold described in panel B as a template for virtual screening and compound selection. For this means, substructure searches were conducted in a database of over 150′000 commercially available compounds, using both the calculated fingerprint and fragments thereof for this purpose. A total of 589 compounds were acquired on the basis of these searches.

The fourth and final step of the process involved testing the acquired compounds in the enzyme assay. Of the 589 compounds selected on the basis of the representative scaffold, 52 molecules showed inhibitory activities of at least 40% when tested in the assay at a concentration of 3 μM. Among these, 12 compounds displayed IC₅₀s in the submicomolar range, and one compound, termed compound D, displayed an IC₅₀ of 65 nM. Six examples of these protease inhibiting molecules are shown below, all of which contain at least one occurrence of the pharmacologically active “fingerprint” shown in panel B:

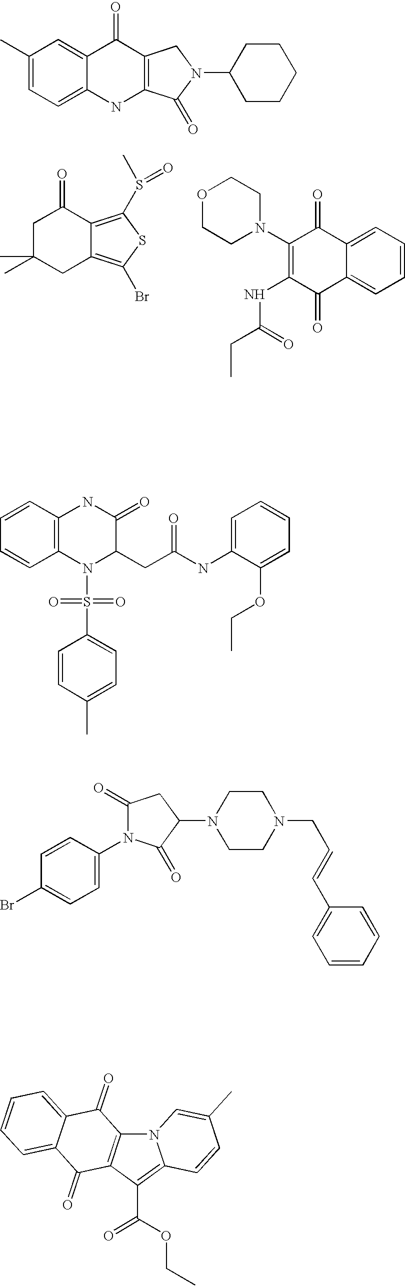

These six protease inhibiting compounds were selected for testing using the method of the present invention. Each molecule significantly inhibited the protein of interest, displaying IC₅₀s in the 0.15 to 15 μM range. As shown by the substructures highlighted in black, the structures of the each of the six compounds contain the pharmacologically active chemical determinant identified using the invention, and shown in panel B above. Some of these compounds actually contain more than one variant of the fingerprint, such as, for example, the tetracyclic structure shown above in the lower right hand corner.

As such, the set of compounds compiled on the basis of the representative fingerprint shown in panel B was 8.7 fold more effective in delivering active molecules than was the originally tested collection of 1680 compounds (p<0.0001). Furthermore, the 52 rationally identified compounds were found to be selective for the protease of interest, insofar as the majority (>90%) failed to show inhibitory activity when tested at a 5 μM concentration on a related protease belonging to the same enzyme family, as well as when tested in the same conditions on 12 other drug targets.

### EXAMPLE NO. 5

### Rational Identification of Novel and Selective Phosphatase Inhibitors

An enzymatic assay was developed for a phosphatase believed to play an important role in receptor sensitization and regulation. A collection of compounds for testing in the assay was assembled, tested, and novel enzyme inhibitors were identified according to the method of the present invention. The first step consisted in generating the necessary structural data for identifying the chemical determinants of inhibitors of the enzyme. This was accomplished by testing the first 12160 compounds of our corporate collection at a 3 μM concentration in the screening assay, and annotating each chemical structure for its inhibitory activity. Using a cutoff of 50% inhibition as a threshold for compound classification, a total of 15 chemical structures were identified as being active, and the remaining 12145 molecules were qualified as inactive.

The second step consisted in identifying the biologically active chemical determinants contained within the structures of the 15 inhibitors. For this means, the 12160 annotated structures were analyzed by selecting the mixed measure of association (VII), wherein x represented the number of active chemical structures containing a chemical determinant of interest, y represented the total number of chemical structures containing the same said chemical determinant, z represented the total number of active chemical structures in the set of N molecules (i.e. z=15), and N represented the total number of chemical structures subject to analysis (i.e. N=12145).

(x/z)−(z−x)/(N−z) (VII)

Measure of association (VII) was then developed into score function (VIII), which the skilled practitioner in the field will recognize as being related to an estimation of a relative risk using the slope of a regression line representing the degree of shared variance that exists between two dichotomous variables, that has been further modified to account for the molecular weight of each chemical determinant under consideration (MW).

Score=MW·e^{[(x/z)-(z-x)/(N-z)]}

In this context, no additional variables other than x, y, z, N, or MW were used in the score function, although it is apparent to the skilled practitioner in the field that formula (VIII) could also be modified to comprise additional variables related to a molecule's material, biological, chemical and/or physico-chemical properties, as mentioned, but not limited to, those cited in example No. 1. The skilled practitioner in the field will also recognize that other measures of association and/or score functions can be used for the same purpose in lieu of those described in formula (VIII), particularly as the comparison of slopes may not, in some instances, allow for sufficient discrimination between two closely-related chemical determinants. The most pertinent of such score functions, in the sense of the present invention, comprise various combinations of two, three, or four of the variables x, y, z and N.

Analysis of the 12160 annotated structures was conducted by scoring a series of chemical determinants with formula (VIII), and retaining structures yielding the largest positive values. This led to the identification of three distinct chemical determinants, ranging from 120 to 220 Da in molecular weight, and having a less than 1 in 10 probability of being contained within the subset of active chemical structures on the basis of chance alone (p<0.1). Accordingly, the three chemical determinants were accepted as being representative of one or more biologically active moieties of the 15 enzyme inhibitors identified in the screen, and were assembled into a fourth list. Calculations using formula (VIII) were then reiterated in order to ascertain whether a larger chemical determinant resulting from the combination, or further expansion, of any of the three fragments could be identified. The largest, statistically significant, chemical determinant found in these additional calculations had a molecular weight of 255 Da, and was selected as a representative scaffold, or pharmacologically active “fingerprint” for subsequent compound selection.

The third step involved using the representative scaffold described above as a template for virtual screening and compound selection. For this means, substructure searches were conducted in a database of over 800′000 commercial and proprietary compounds using both the calculated fingerprint and fragments thereof for this purpose. A total of 1242 compounds were selected for testing on the basis of these searches, and the same collection of 1280 randomly selected compounds described in example no. 1 was used for control purposes.

The fourth and final step of the process involved testing the compounds in the enzyme assay. Of the 1242 compounds selected on the basis of representative scaffolds, 34 molecules showed inhibitory activities of at least 50% when tested at a concentration of 3 μM. Among these, eight compounds displayed IC₅₀s in the submicromolar range, and one compound, termed compound E, displayed an IC₅₀ of 87 nM (FIG. 14).

FIG. 14 illustrates the effect of compound E on phosphatase-dependent protein dephosphorylation. The phosphatase of interest was incubated with phosphorylated peptide substrate in the presence of increasing concentrations of compound E. Substrate dephosphorylation was assayed by measuring the release of free phosphate into the reaction medium with malachite green. Compound E significantly inhibited phosphatase dependent dephosphorylation, displaying an IC₅₀ of 87 nM.

Among the 1280 randomly selected compounds tested for control purposes, only two showed inhibitory activity in the screening assay, the most potent of which displayed an IC₅₀ of only 1.8 μM. As such, the set of compounds compiled on the basis of representative fingerprints was 17.5 fold more effective in delivering active molecules than was the set of randomly selected compounds (p<0.0005), and 22.3 times more effective than the first 12160 compounds of the corporate compound collection (p<0.00001 ).

Finally, compound E was found to represent a novel, hitherto unreported, class of phosphatase inhibitor, showing greater than 20-fold selectivity for the target of interest when tested in selectivity assays using both structurally- and functionally-related, alternative phosphatases.

### EXAMPLE NO. 6

### Increasing the Potency of a Chemical Series

The invention can also be used for increasing the potency of a chemical series. Exemplifying this, a collection of 1251 compounds was tested at a 3 μM concentration in a protease assay, which yielded 25 compounds displaying inhibitory activities of at least 40%. Analysis of the structures was performed as described in example No.1, which led to the identification of a number of chemical determinants, one of which had less than a 1 in 10′000 probability of occurring among 7 of the 25 protease inhibitors on the basis of chance alone (p<0.0001). Unfortunately, the seven compounds containing this determinant only displayed moderate inhibitory activities (mean IC₅₀=3.4 μM±1.34 μM, n=7), making them unattractive for chemical follow-up. Consequently, the determinant in question was accepted as representing the biologically active moiety of the inhibitors of interest, and was directly used as a representative scaffold, or pharmacologically-active “fingerprint”, for additional compound selection.

For this means, a database of over 100′000 commercially available molecules was screened for the determinant of interest, and 142 molecules were selected for additional testing. Among these 142 compounds, 11 showed inhibitory activities in the submicromolar range, displaying a mean IC₅₀ of 0.48 μM±0.09 μM (n=11, mean IC₅₀ significantly smaller than previous value at p<0.05). As such, the method of the present invention allows one to significantly increase the pharmacological potency of a chemical series.

### EXAMPLE NO. 7

### Increasing the Selectivity of a Chemical Series

The invention can also be used for increasing the selectivity of a chemical series. Exemplifying this, a collection of 3360 compounds was tested at a 3 μM concentration in a kinase assay, termed kinase assay No. 1, which yielded 22 compounds displaying inhibitory activities of at least 40%. Analysis of the structures was performed as described in example No. 2, which led to the identification of a number of chemical determinants, one of which, termed “determinant No. 10”, was estimated as having approximately less than a 1 in 20 probability of occurring among 3 of the 22 kinase inhibitors on the basis of chance alone (p<0.05). Unfortunately, selectivity assays performed on four other kinases revealed that determinant No. 10 was also an important constituent of inhibitors of another kinase, termed kinase No. 2, suggesting that selective inhibitors of kinase No. 1 could not be developed on the basis of determinant No. 10 alone. Indeed, the three structures containing determinant No. 10 were equipotent on the two kinases, displaying mean IC₅₀s of 7.2 μM±3.81 μM (n=3), and 21.5 μM±9.29 μM (n=3) on kinases No. 1 and 2, respectively, which represented a selectivity ratio of only 2.98 in favor of kinase No. 1.

In this view, the 3360 compounds tested on kinase No. 1 were retested at a 3 μM concentration on kinase No. 2, which yielded 92 compounds displaying inhibitory activities of at least 40%. The list of 3360 structures was subsequently annotated for both kinase No.1 and No. 2 activities, and analysis was performed according to the method of the present invention by selecting measure of association (III), and developing it into score function (IX), wherein x₁ represented the number of chemical structures active on kinase No.1 containing a chemical determinant of interest, x₂ represented the number of chemical structures active on kinase No. 2 containing the same said chemical determinant, y represented the total number of chemical structures containing the chemical determinant, z, represented the total number of chemical structures active on kinase No. 1 in the set of N molecules (i.e. z₁=22), z₂ represented the total number of chemical structures active on kinase No. 2 in the set of N molecules (i.e. Z₂=92), and N represented the total number of chemical structures subject to analysis (i.e. N=3360).

$$( IX ) Score = \frac{{x}_{1} ( N - y - {z}_{1} + {x}_{1} ) ( {z}_{2} - {x}_{2} ) ( y - {x}_{2} )}{{x}_{2} ( N - y - {z}_{2} + {x}_{2} ) ( {z}_{1} - {x}_{1} ) ( y - {x}_{1} )}$$

The skilled practitioner in the field will recognize score function (IX) as a way to compare relative risks, allowing one to identify the chemical determinants that are most likely to be selective for one kinase over the other. In this context, it is apparent to the skilled practitioner that formula (IX) could be modified to comprise additional variables related to a molecule's material, biological, chemical and/or physico-chemical properties, as mentioned, but not limited to, those cited in example No. 1. Finally, it is also recognized that other measures of association and/or score functions can be used for the same purpose in lieu of those described in formulas (III) and (IX). For example, measure of association (I) could be used in score function (II), and the resulting score values for kinase No. 2 activity could be subtracted from those obtained for kinase No. 1 activity, or conversely, the values obtained for kinase No.1 activity could be divided by those obtained for kinase No. 2. Numerous other approaches are also possible, the most pertinent of which, in the sense of the present invention, employ score functions comprising various combinations of two, three of four of the variables x, y, z and N.

Scoring a series of chemical determinants with formula (IX) led to the identification of a number of kinase No. 1 selective chemical determinants, one of which, termed “determinant No. 11”, consisted of determinant No. 10 substituted with an additional chemical motif. Consequently, determinant No. 11 was accepted as representing a pharmacologically active moiety of selective inhibitors of kinase No. 1, and was used as a representative scaffold, or pharmacologically active “fingerprint”, for subsequent compound selection. For this means, substructures searches were conducted in a database of over 400′000 commercially available compounds using determinant No. 11 and fragments thereof. A total of 498 compounds were acquired on the basis of these searches, which after testing in the two assays, yielded three inhibitors containing determinant No. 10, and displaying mean IC₅₀s of 0.94 μM±0.52 μM (n=3), and 31.6 μM±4.41 μM (n=3) in kinase assays No. 1 and 2, respectively. This result represents an 11-fold increase in the selectivity ratio of the series for kinase No. 1 over kinase No. 2 (from 2.98 to 33.6, p<0.05), demonstrating that the method of the present invention allows one to increase the pharmacological selectivity of a chemical series of interest.

### EXAMPLE NO. 8

### Rational Identification of Series with Multiple Pharmacological Effects

A functional assay was developed for a ligand-gated ion channel believed to play a role in the immune response. A collection of compounds for testing in the assay was assembled, tested, and novel ion channel blockers were identified according to the method of the present invention. The channel under investigation was described as belonging to a family of targets that were permeant to sodium ions, activated by purine nucleotides, and inhibited by a certain sodium channel blockers. In this light, it was decided to identify pharmacological fingerprints having the dual capacity of mimicking purine nucleotides and inhibiting sodium channels at the same time, in view of increasing the chances of rapidly identifying inhibitors of the ligand-gated ion channel of interest.

The first step of the process comprised the compilation of two lists of chemical structures by reviewing the current literature. The first list contained the structures of 79 documented sodium channel inhibitors. The second contained the structures of 2367 inhibitors of purine-nucleotide binding proteins (see example No. 2 for details). The second step of the process consisted in identifying the biologically active chemical determinants simultaneously contained with in both lists of chemical structures. For this means, each list was supplemented with the structures of more than 100′000 molecules described as having no effect on the surrogate target(s) of interest, and the analysis was conducted by selecting subtractive measure of association (I), as described in example No. 1., and developing it into score function (X), wherein x₁ represented the number of chemical structures active at sodium channels and containing a chemical determinant of interest, x₂ represented the number of chemical structures active at purine nucleotide- binding proteins and containing the same said chemical determinant, y₁ represented the total number of structures containing the chemical determinant in the list of structures annotated for sodium channel blocking effects, y₂ represented the total number of structures containing the chemical determinant in the list of structures annotated for purine nucleotide-binding protein inhibition, z₁ represented the total number of structures inhibiting sodium channels in the set of N₁ molecules (i.e. z₁=79), z₂ represented the total number of chemical structures acting at purine nucleotide binding proteins in the set of N₂ molecules (i.e. z₂=2367), and N₁ and N₂ represented the total number of chemical structures subject to analysis in the respective lists of annotated structures.

$$( X ) Score = \frac{1}{\sqrt{2}} ( \sqrt{\frac{{( {N}_{1} {x}_{1} - {y}_{1} {z}_{1} )}^{2} {N}_{1}}{{z}_{1} ( {N}_{1} - {z}_{1} ) {y}_{1} ( {N}_{1} - {y}_{1} )}} + \sqrt{\frac{{( {N}_{2} {x}_{2} - {y}_{2} {z}_{2} )}^{2} {N}_{2}}{{z}_{2} ( {N}_{2} - {z}_{2} ) {y}_{2} ( {N}_{2} - {y}_{2} )}} )$$

The skilled practitioner in the field will recognize score function (X) as a way to combine two different tests of association, allowing one to identify the chemical determinants that are most likely to have effects on both sodium channels and purine nucleotide-binding proteins at the same time. In this context, is apparent to the skilled practitioner that formula (X) could be modified to comprise additional variables related to a molecule's material, biological, chemical and/or physico-chemical properties, as mentioned, but not limited to, those cited in example No. 1. It is also recognized that other measures of association and/or score functions can be used for the same purpose in lieu of those described in formulas (I) and (X), particularly as score function (X) does not take into account the direction of the differences existing between the proportions of the two data sets, all the while requiring that these proportions be comparable, and further more, that N₁ be comparable to N₂, and that both values be larger than 20. For example, one may wish to weight results for data sets where sample sizes are considerably different by using a score function based on a weighted mean of the difference between proportions (see example 21 further on). Alternatively, one may want to include a third, or fourth, or ith pharmacological property into the calculation, in which case it is apparent that formula (X) can be extended to its more general form (XI), wherein d represents the number of compound lists undergoing analysis, and where the resulting score values can be directly referred to tables of the standard normal distribution in order to determine the likelihood of having found one or more chemical determinants that are at the basis of all the pharmacological properties under consideration. Numerous other approaches are also possible, the most pertinent of which, in the sense of the present invention, employ score functions comprising various combinations of two, three of four of the variables x, y, z and N.

$$Score = \frac{1}{\sqrt{d}} {\sum }_{i = 1}^{d} {( \sqrt{\frac{{( Nx - yz )}^{2} N}{z ( N - z ) y ( N - y )}} )}_{i} \qquad ( XI )$$

Analysis of the two lists of annotated structures was conducted by scoring a series of chemical determinants with formula (X), and by retaining the structures yielding the largest values bigger than 2. This led to the identification of a chemical determinant having less than a 1 in 20 probability of occurring in both subsets of biologically active structures on the basis of chance alone (p<0.05). Accordingly, the chemical determinant, termed “determinant No. 12”, was accepted as being representative of one or more biologically active moieties of both sodium channel and purine nucleotide-binding protein inhibitors, and was directly used as a representative scaffold, or pharmacologically active “fingerprint” for subsequent compound selection.

The third step of the process involved using the representative scaffold as a template for virtual screening. For this means, substructure searches were conducted in a database of over 250′000 commercially available compounds using determinant No. 12 and fragments thereof for this purpose. A total of 800 compounds were acquired on the basis of these searches, and the same collection of 1280 randomly selected compounds described in example No. 1 was used for control purposes.

The fourth and final step of the process involved testing the acquired compounds in the ion channel assay. Of the 800 molecules selected on the basis of determinant No. 12, twenty three compounds showed inhibitory activity of at least 40% when tested at a concentration of 3 μM. Among these, three compounds displayed IC₅₀s in the submicromolar range, and one compound, termed compound F, displayed an IC₅₀ of 145 nM±56 nM (n=4). Among the 1280 randomly selected compounds tested for control purposes, only one molecule displayed significant inhibitory activity in the low micromolar range, and its chemical structure actually contained a substantial portion of determinant No. 12. Interestingly, when the same collection of 800 compounds was tested in on a kinase that is also believed to play a role in the immune response, eight compounds showed inhibitory activities of at least 40% when tested at 5 μM, compound F displayed an IC₅₀ of 1.2 μM, and another compound, termed compound G, displayed an IC₅₀ of 137 nM±48 nM (n=4). Compounds F, G, and a number of closely-related molecules also containing determinant No. 12 in their structures were further found to inhibit sodium channels, typically displaying 50-100% inhibitions at 1 μM. Taken together, these results demonstrate that the method of the present invention allows one to select and/or design compounds with multiple pharmacological properties, which may be of interest for developing drugs for use in the treatment multifactorial disease states, such as, but not limited to, inflammation. It is also apparent that, by analogy, the method can be used to incorporate novel pharmacological properties into a chemical series previously devoid of such said properties.

### EXAMPLE NO. 9

### Compiling Lists of Biologically Active Chemical Determinants

In a preferred embodiment of the present invention, the method can also be used for compiling lists of biologically active chemical determinants, which in turn can be employed as reference databases for use in the conduct of rational drug design, such as, for example, in a computer-controlled decision making programs for use in medicinal chemistry. Exemplifying this, the scientific literature was reviewed, and 25 lists of pharmacologically active molecules were assembled, each list comprising the chemical structures of compounds displaying a given pharmacological property, such as, for example, sigma receptor binding, dopamine D₂ receptor agonism, and estrogen receptor antagonism. Each list was subsequently analyzed according to the invention by selecting measure of association (III), as described in example No. 2, and developing it into function (IV), which was used to score various chemical determinants contained with in one or more of the lists undergoing analysis. These calculations led to the identification of a large number of pharmacologically active chemical determinants, three of which are shown in a portion of the resulting matrix in the following table:

| | Sigma | | Estrogen |
|---|---|---|---|
| Determinant | Ligand | D₂ Agonist | antagonist |
| 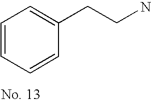 | 1.85 | 8.12 | 0.05 |
| 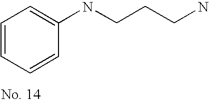 | 2.40 | 0.00 | 0.00 |
| 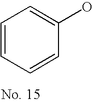 | 0.91 | 2.93 | 28.17 |

This table provides a reference list of pharmacologically active chemical determinants. Twenty five lists of structures containing molecules described as having one of twenty five different pharmacological properties were assembled, and analyzed according to the method of the present invention using measure of association (III) and score function (IV). The twenty five properties included the capacity to bind to sigma receptors (sigma ligand), dopamine D₂ receptor agonism (D₂ agonist), and estrogen receptor antagonism (estrogen antagonist). A small portion of the resulting 26 column matrix is shown in the table above. Values greater than 1 indicate that a given chemical determinant has less that a 1 in 20 probability of occurring by chance in a set of molecules sharing the same pharmacological property, indicating that the determinant is most likely to be at the molecular basis of the same said property. Tables such as the one shown above constitute repositories of biologically active determinants, or “fingerprints”, which can be used as reference lists for making informed decisions in drug discovery and development.

Interpretation of the resulting table is conducted as follows. Compounds whose chemical structures contain determinant No. 13 are more likely to display dopamine D₂ receptor agonist properties than either sigma receptor binding or estrogen receptor antagonist properties, as 8.12>1.85>0.05. Conversely, determinant No. 13 is a preferred determinant for constructing collections of potential dopamine D₂ receptor agonists, as 8.12>2.93>0.00. In the same way, compounds whose chemical structures contain determinant No. 14 are more likely to be sigma receptor ligands than either dopamine receptor agonists or estrogen receptor antagonists, as 2.4>0.00=0.00. Again, determinant No. 14 is the preferred determinant for compiling sets of sigma receptor ligands, as 2.40>1.85>0.91. Finally, compounds whose chemical structures contain determinant No. 15 are most likely to exhibit estrogen receptor inhibiting properties, as 28.17>2.93>0.91, and alternatively, determinant No. 15 is the preferred fingerprint for compiling collections of potential estrogen receptor antagonists, as 28.17>0.05>0.00.

It is apparent to the skilled practitioner in the field that other measures of association and/or score functions could be used for constructing such tables in lieu of those described in formulas (III) and (IV). It is also recognized that the score function employed could comprise additional variables related to a structure's material, biological, chemical and/or physico-chemical properties, as mentioned, but not limited to, those cited in example No. 1. It is further apparent that the score function or the scoring process could also be modified to comprise a weighting or normalization step in order to make individual score values more readily comparable with each other, which is certainly the case in the above table, three similar sized samples were used in its construction, but may not be the case for other data sets. Finally, it is apparent that the same process can be used to compile reference lists of structures scored for other properties of interest in discovery process, such as, but not limited to, general therapeutic use, toxicity, absorption, distribution, metabolism, and/or excretion.

### EXAMPLE NO. 10

### Predicting the Secondary Pharmacological Actions of a Molecule

The invention can further be used to predict the secondary actions of a molecule. Illustrating this, a novel class of ion channel blockers was identified as shown in example No. 3. As previously described for other inhibitors of this same channel, the basic chemical structure of the new chemical series of inhibitors contained the chemical determinant shown in panel B of example No. 3, notably in the form of determinant No. 5 shown in panel A of example No. 3. By comparing determinant No. 5 to the determinants contained in the above table, it was projected that the inhibitors of interest had a very high chance of binding to sigma receptors, particularly as the chemical structure of determinant No. 5 is identical to that of determinant No. 14. Consequently, channel blockers containing determinant No. 5 were tested in sigma σ₁ and σ₂ receptor binding assays, and found to exhibit submicromolar affinities for both sites. As such, these results demonstrate that the score values obtained using the method of the present invention allow one to predict the secondary actions of a chemical series, which is extremely useful for series progression in medicinal chemistry.

### EXAMPLE NO. 11

### Identification and Prediction of the Toxic Actions of a Molecule

It is clear from the preceding examples that the method of invention can also be used to identify toxicophoric chemical determinants contained within pesticides, herbicides, insecticides, and the like, and this simply by analyzing lists of structures that are annotated for toxicological instead of pharmacological properties. In this context, the invention can be directly applied to the identification of more potent, selective and/or more broadly-acting toxic chemical series for use in, for example, agricultural chemistry programs for crop protection.

Alternatively, the invention can be used to compile reference lists, or databases, of toxic chemical determinants in a manner identical to that described in example No. 9. Such lists can then be used for estimating the likelihood that a chemical series will exhibit a given toxic effect, which is of use, for example, in the screening of food additives and environmental chemicals.

Illustrating the possibility of predicting toxic effects in the pharmaceutical research setting, 4480 compounds were tested on a cellular phosphatase of interest for the treatment of inflammation. A total of 25 compounds showed inhibitory activities of at least 40% when tested at 10 μM in the assay, all of which displayed IC₅₀s in the low micromolar range. Results analysis conducted according to the method of the present invention, which led to the identification of two molecularly distinct chemical determinants most likely to be at the basis of pharmacological activity, termed determinants No. 16 and 17. As the two determinants were present in equipotent molecules, and both were felt to be able to yield chemical series that would be equally amenable to chemical follow-up, it was decided to select between the two on the basis of predicted toxic side effects.

For this means, the structures of determinants No. 16 and 17 were compared to structures contained in a toxicological database, and it was found that molecules containing determinant No. 16 in their structures had a significantly higher likelihood of being cytotoxic than compounds containing only determinant No. 17. This indicated that phosphatase inhibitors bearing determinant No. 16 would be less interesting for progression due to inherent cytotoxicity of the pharmacological fingerprint. This hypothesis was verified experimentally by exposing cultured cells to 1 μM concentrations of both classes of inhibitor, and by measuring cell viability using a standard MTT assay, where it was found that all compounds containing determinant No. 16 induced cell death within 24 hours of application, which was not the case for the majority of compounds bearing determinant No. 17. As such, these results clearly demonstrate that the method of the present invention allows one to identify and/or predict chemical series that are most likely to exhibit toxic properties in a given setting. In this context, it is apparent that identical calculations can be performed using, for example, mutagenicity data (Ames tests), P450 isozyme inhibition data, or data derived from any other relevant toxicity test.

### EXAMPLE NO. 12

### Identification of the Biologically Active Moieties of Receptor Ligands

A cell surface receptor was selected as a target of interest for the control of certain endocrine disorders. The receptor was described as being endogenously activated by a nonapeptide hormone produced by the pituitary gland. A list of chemical structures described as being ligands of the same said receptor was compiled by reviewing the scientific literature. The list was subsequently analyzed according to the method of the present invention, using measure of association, score function (IV), and a list of chemical determinants comprised of fragments of the twenty common amino acids (glycine, alanine, valine, leucine, isoleucine, proline, serine, threonine, tyrosine, phenylalanine, tryptophan, lysine, arginine, histidine, aspartate, glutamate, asparagine, glutamine, cysteine and methionine), supplemented by fragments of the peptide backbone structure (NH—CH—CO—)₃. Examples of these determinants are shown below:

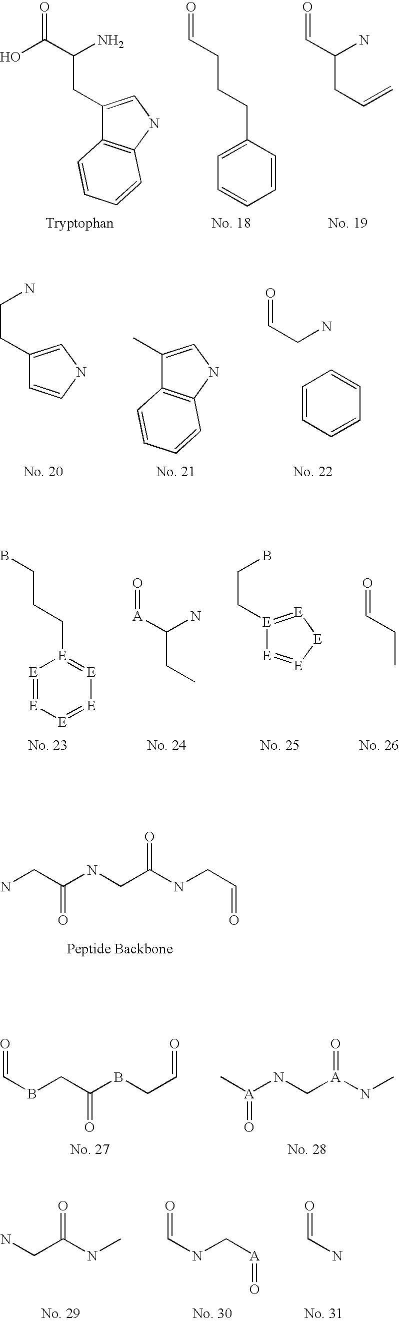

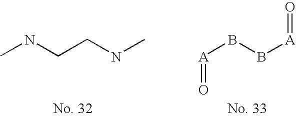

These are examples of amino acid and peptide backbone-derived chemical determinants used for analysis. A list of receptor ligands was compiled by reviewing the scientific literature, and analyzed according to the invention using measure of association (III), score function (IV), and a list of chemical determinants comprised of various fragments of the twenty common amino acids supplemented by fragments of the peptide backbone structure (—NH—CH—CO—)₃. Examples of some of the determinants derived from tryptophan are shown in the first two rows. These were either exact fragments (ex: determinants No. 18, 19, 20, 21 and 26), assemblies of exact fragments (ex: determinant No. 22), inexact fragments (ex: determinants No. 23, 24 and 25), or assemblies of exact and inexact fragments (not shown). Lower two rows: examples of determinants derived from the peptide backbone structure (NH—CH—CO—)₃, representing exact (determinants No. 29, 31, 32) and inexact fragments (determinants No. 27, 28, 30, 33). ). Symbols: A represents C or S; B represents C or N; E represents C, N, O or S.

Scoring the fragments with formula (IV) led to the identification of a number of chemical determinants having score values greater than 1, indicating that the corresponding structures had less than a 1 in 20 probability of being contained within the subset of pharmacologically active compounds on the basis of chance alone (p<0.05). Examples of such determinants are shown below, along with their respective score values:

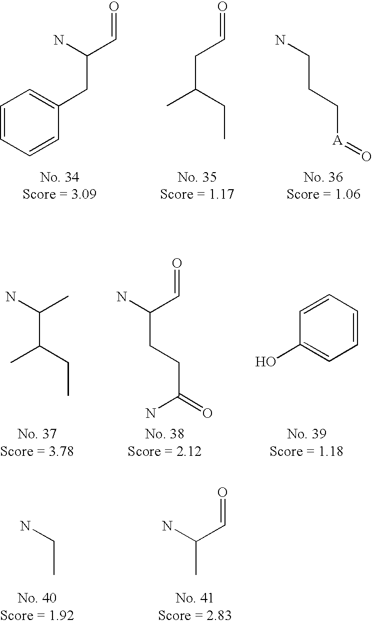

These are examples of high-scoring chemical determinants identified in first round of analysis. A collection of receptor ligands was analyzed according to the present invention by scoring the chemical determinants shown before, as well as a number of others, with score function (IV). Values greater than one indicated that the determinant had less than a 1 in 20 probability of occurring in the subset of receptor ligands on the basis of chance alone. The figure above shows some of the higher scoring chemical determinants that were identified in this process.

Accordingly, these determinants were accepted as being representative of one of more amino acids contained within the primary sequence of the peptide hormone, and were assembled into a second list. Calculations using formula (IV) were then reiterated in order to identify the highest scoring combinations of these new determinants, a number of which obtained score of values greater than 10. The structure of the highest ranking chemical determinant, termed determinant No. 42, was subsequently compared to the structures of the 800 dipeptides comprised of various combinations of 20 amino acids, and it was determined that only one dipeptide sequence, termed A₁-A₂, contained determinant No. 42 in its entirety. This result was taken to indicate that the hormone of interest most likely comprised the A₁-A₂ sequence somewhere within its primary structure, and further more, that at least one of the two amino acids played an important role in the binding of the endogenous ligand to its receptor. Verification of the sequence of the hormone revealed that it did indeed comprise the predicted A₁-A₂ sequence, an event that was calculated as having a probability of only 0.019 of occurring on the basis of chance alone. Interestingly, other work showed that peptides containing a mutation in the A₂ position of the A₁-A₂ sequence (e.g. A₁-A₃, or A₁-A₄ instead of A₁-A₂, where A₁, A₂, A₃ and A₄ are different amino acids) exhibited a markedly lower affinity for the receptor, illustrating that at least one of the two predicted residues did indeed constitute an important moiety underlying the biological function of hormone of interest. Taken together, these results demonstrate that the method of the present invention allows one to identify the biologically active moieties of peptide ligands, which is useful in medicinal chemistry programs focussing on the rational design of, for example, peptidomimetic enzyme inhibitors and/or receptor ligands.

### EXAMPLE NO. 13

### Prediction of Protein-Protein Interactions

The invention also allows one to predict the existence of protein-protein interactions in a manner analogous to that described in the preceding example. Illustrating this, an ion channel screen was implemented as described in example No. 3, which led to the identification of more than two dozen molecules displaying at least 40% inhibition when tested at a concentration of 5 μM. The chemical structures of these inhibitors were assembled into a list, which was analyzed as described in example No. 12. This led to the identification of a series of high-scoring, amino acid and peptide backbone-derived chemical determinants, which after further analysis, were found to indicate that the channel of interest was most likely to interact with an inhibitory peptide or protein specifically containing a certain dipeptide sequence, termed A₅-A₆. Interestingly, such inhibitory proteins had previously been described in the literature, all of which contained a 20 amino acid “channel inhibiting” domain containing exactly the predicted A₅-A₆ dipeptide sequence. As it can be determined that any 20 amino acid sequence has a probability of only 0.046 of containing a given sequential arrangement of two given residues on the basis of random chance, it can be estimated that the probability of correctly predicting the existence of two distinct dipeptide sequences existing in two unrelated proteins on the basis of chance in this and in the preceding example is less than 1 in 1097. Nevertheless, the correct predictions were made in both cases, demonstrating that the invention allows one to identify and/or predict existence of certain types of protein-protein interactions. This can be done simply by identifying the sequence of amino acids containing the largest possible chemical determinant identified from within the subset of pharmacologically active structures, and then searching in sequence databases for proteins containing the amino acid sequence of interest. A description of this process is supplied in example No. 14 below. In this context, it is apparent to the skilled practitioner that the approach is not limited to the sole identification of dipeptide sequences, as depending on the structures of the pharmacologically active compounds undergoing analysis, tri- or even tetrapeptide sequences could also be detected. It is also apparent that a similar approach could also be used for non-peptide ligands, that is, that the method could also be adapted for the detection of, for example, carbohydrate sequences (i.e. sugars), nucleotides, and the like.

### EXAMPLE NO. 14

### Identification of Orphan Ligand-Receptor Pairs

The invention can further be applied to the identification of orphan ligands and/or orphan ligand-receptor pairs. The process is initiated by compiling a list of chemical structures having a given effect on a protein of interest (typically binding), but for which no ligands are known at the time of investigation. This information can be generated in a number of ways, such as, but not limited to, conducting of NMR studies, measuring conformational changes by circular dichroism, measuring protein-ligand interactions by surface plasmon resonance, or in the case of an orphan receptor, by performing assays with constitutively-activated mutants of the receptor of interest.

Illustrating this concept, let us suppose that experiments of the type described above are conducted on an orphan receptor, yielding the structures shown below:

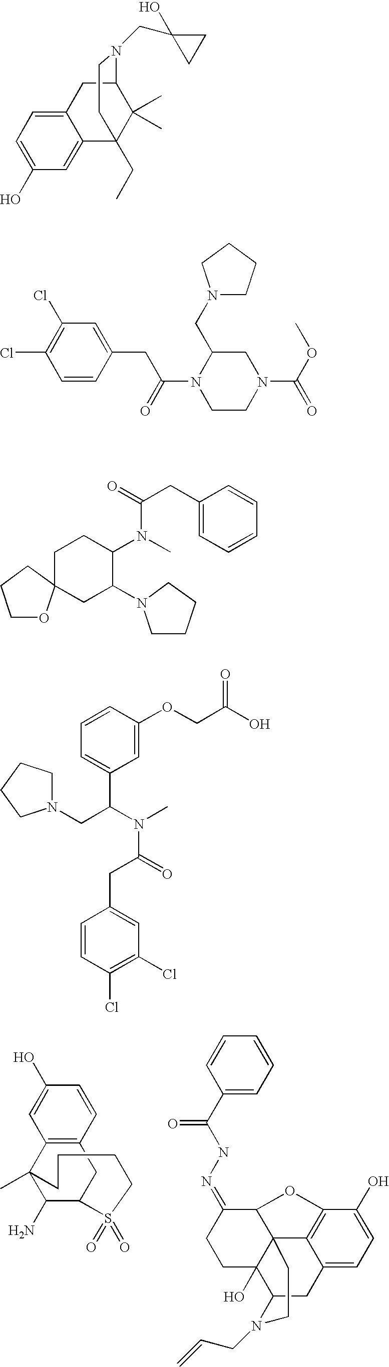

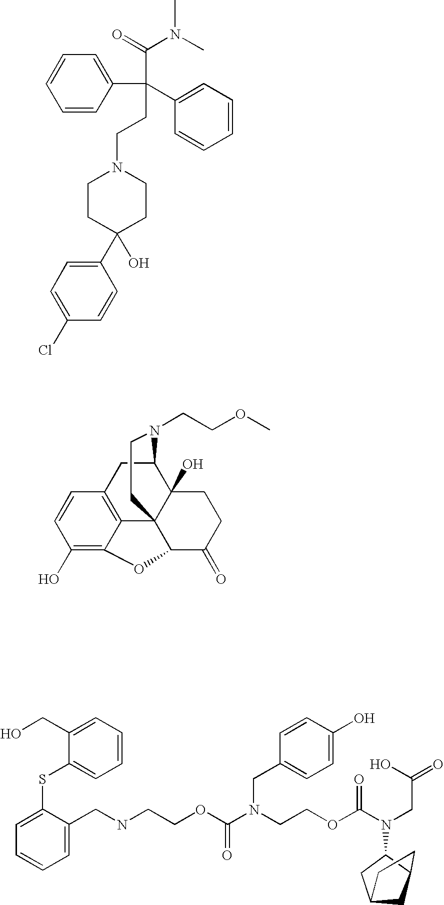

This is a hypothetical list of structures analyzed for biologically active chemical determinants. The nine structures shown above were analyzed according to the invention as described in example No. 12, using the aforementioned list of amino acid and peptide backbone-derived chemical determinants.

Analysis of the structures as described in example No. 12 leads to the identification of a number of amino acid and peptide backbone-derived chemical determinants with scores larger than 1. Examples of such determinants are shown below, along with their corresponding score values:

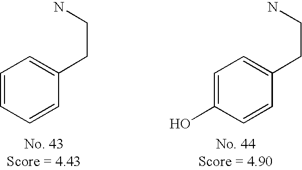

These are examples of high-scoring chemical determinants identified in first round of analysis. The collection of hypothetical receptor ligands was analyzed according to the invention by scoring the chemical determinants shown in the first panel of example No. 12, as well as a number of others, with score function (IV). Values greater than one indicated that the determinant had less than a 1 in 20 probability of occurring in the subset of ligands on the basis of chance alone. Shown above are two of the higher scoring chemical determinants that were identified in this process.

It is clear from these examples that determinants No. 43 and 44 can only be contained within the chemical structures of the amino acids phenylalanine and tyrosine. As such, it is inferred that peptides that interact with the orphan receptor are likely to contain either a tyrosine or phenylanine residue with in their sequences, and that these residues are likely to play an important role in either the binding of the ligand(s) and/or the activation of the receptor by these peptide(s). If high-scoring determinants No. 43 and 44 are subsequently reanalyzed in order to ascertain whether combinations with fragments of other amino acids do not yield even higher scoring structures, fragments such as determinant No. 45, shown in the following panel A, can be further identified.

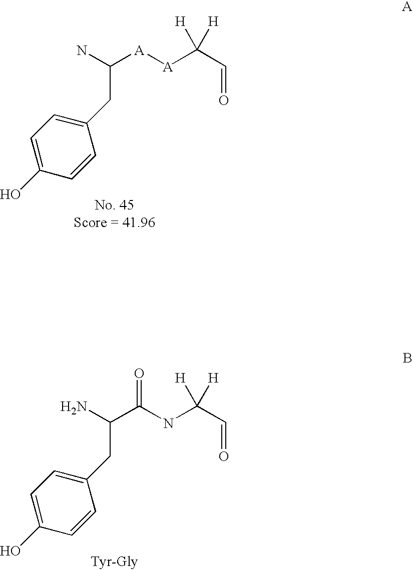

These panels show high-scoring chemical determinants identified in second round of analysis. Chemical determinants such as those described before were reanalyzed according to the invention to determine whether combinations with fragments of other amino acids would not produce still higher scoring structures. One of these, termed determinant No. 45 (Panel A), displayed a score value greater than 40. Interestingly, the entirety of determinant No. 45 is contained in the structure of the dipeptide sequence Tyr-Gly (Panel B), inferring that an endogenous ligand of the orphan target of interest contains a Tyr-Gly dipeptide sequence within its primary structure.

As it is clear that the entirety of determinant No. 45 is contained within the structure of the dipeptide tyrosine-glycine (Tyr-Gly), it is inferred that the orphan ligand(s) that we are looking for are most likely to contain a Tyr-Gly sequence somewhere within their primary structures. On the basis of this information, databases of amino acid sequences can be screened in order to identify known and/or orphan ligands containing the predicted Tyr-Gly sequence, which after selection and expression, can be tested in the original biochemical screening assay. Alternatively, chemical determinant No. 45 can be directly used to compile compound collections of potential Tyr-Gly mimetics.

Finally, it is worth noting that the chemical structures used in this example are actually opioid receptor agonists taken from the literature, and that the naturally-occuring opioid receptor agonists dynorphin A, β-endorphin, leu-enkephalin and met-enkephalin all contain the predicted Tyr-Gly sequence in their primary structures. As the tyrosine residue has been shown to be absolutely required for opiod agonist activity, the current example further illustrates the capacity of the invention to identify biologically-active moieties of receptor ligands. It is also recognized that the estimations described above can be improved by using alternative algorithms employing the variables x, y, z and N, such as, for example, in Fischer's exact test. Indeed, only nine structures were analyzed by using a method for which no adequate correction for small sample sizes was made, suggesting that the score value of 41.96 for determinant No. 45 may be somewhat overestimated.

### EXAMPLE NO. 15

### Identification of Endogenous Modulators of Drug Targets

It is apparent to the skilled practitioner that the invention can also be applied to the identification of endogenous modulators of drug targets. Exemplifying this, a functional assay was developed for an ion channel of interest in the treatment of neurodegeneration. A compound collection was screened, and the resulting list of inhibitors was analyzed for the presence of biologically active chemical determinants as described in example No. 2. This led to the identification of a high scoring chemical determinant which was found to be contained with in a subset of molecules endogenously produced in eukaryotic cells. The corresponding compounds were subsequently purchased and tested in the assay, where it was found that the channel of interest was selectively inhibited by submicromolar concentrations of a particular subclass of cellular phospholipid, which most interestingly, had previously been associated with neuronal apoptosis through an unknown mechanism by other groups. Taken together, these results demonstrate that the invention allows for the identification of endogenous modulators of drug targets.

### EXAMPLE NO. 16

### Identification of False Positive Experimental Results

An enzymatic assay was developed for a protein kinase believed to play an important role in the immune response. A compound collection for screening on the target was assembled according to the invention, notably as described in example No. 2. The compounds of the collection were subsequently tested in the assay at a concentration of 5 μM, which led to the identification of 35 molecules displaying inhibitions of at least 40%. The structures of these compounds were analyzed using a simplified variant of formula (II) as a score function, and the corresponding score values were directly compared to those of a statistical table, which provided estimations of the probabilities that given chemical determinants occurred among the subset of 35 pharmacologically active compounds on the basis of chance alone.

Using a threshold for the probability of chance occurrence of p<0.05, it was determined that 14 of the 35 inhibitors were most likely to represent false positive results. Subsequent retesting of the 14 compounds in the assay confirmed this hypothesis, illustrating that the invention allows for the identification of false positive experimental results.

### EXAMPLE NO. 17

### Identification of False Negative Experimental Results

By performing calculations analogous to those described in example No. 16, the invention further allows for the identification of false negative experimental results. Exemplifying this, the chemical structures of a series of phosphatase inhibitors were analyzed for the presence of pharmacologically active chemical determinants as described in example No. 16. The resulting, highest scoring chemical determinants were used as pharmacologically active “fingerprints” for performing substructure searches in the list of chemical structures corresponding to the compounds that were originally tested in the assay. This revealed a number of molecules that contained one or more of the aforementioned chemical determinants, but which were nevertheless identified as being negative in the screening assay. The corresponding molecules were subsequently retested in the assay, where it was found that more than 15% of these were false negatives, one compound even displaying submicromolar inhibitory activity. These results clearly demonstrate that the method of the present invention allows for the identification of false negative experimental results.

### EXAMPLE NO. 18

### Conducting Quantitative Configurational and Conformational Analyses

In a further improved embodiment of the invention, one can also employ algorithms comprising various combinations of the variables x, y, z and N for quantitative conformational and/or configurational analysis. Illustrating this possibility, it is clear from the results shown in example No. 4 that the structure of the pharmacologically active, protease-inhibiting “fingerprint” shown in panel B of example No. 4 is neither configurationally nor conformationally defined. Indeed, it is impossible to tell from the representation of the structure whether, in relation to the two carbonyl or sulfonyl groups, it is the trans-oid or cis-oid conformation of the single bond version of the fingerprint that is pharmacologically active, or furthermore, whether it is the (E) or (Z) configuration of the fingerprint that is active in the case of the double bond version of the same said structure. The reason for this is that the calculations performed in example No. 4 were directed towards identifying the chemical determinant that was most likely to be at the basis of protease-inhibiting activity, without considering the possible conformations and/or configurations that such a determinant may take. In view of the fact that numerous pharmacologically active structures contain double bonds and/or ring systems, which serve to conformationally constrain chemical determinants by reducing their total number of rotatable bonds, it is possible to use the invention to determine which conformations and/or configurations of a given chemical determinant are most likely to be pharmacologically active.

Exemplifying this, the six (protease inhibiting) structures shown in example No. 4 were analyzed by scoring a series of conformationally and configurationally-defined chemical determinants derived from the structure shown in panel B of example No. 4, with score function (IV).

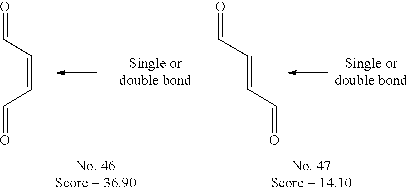

This panel illustrates the quantitative conformational/configurational analysis of a protease-inhibiting chemical determinant. The six structures shown in example No. 4 were analyzed according to the invention using a list of conformationally- and configurationally-defined chemical determinants.

Chemical determinant No. 46, shown along side lower scoring chemical determinant No. 47 above, obtained one of the highest score values, inferring that the (Z) configuration of the double bond version of the fingerprint is more likely to be the preferred arrangement contained in the chemical structures of inhibitors of the protease of interest. This hypothesis was subsequently verified by further focused highthroughput screening, which delivered numerous protease inhibitors in which the pharmacologically active fingerprint was indeed constrained in the (Z) or “cisoid”configuration, and only very few where it was not.

Taken together, these results demonstrate that the method of the present invention allows for the identification of the biologically active conformations and/or configurations of chemical determinants. Finally, it is recognized that such calculations can be performed with a number of alternative algorithms employing various combinations of the variables x, y, z and N. In this context, it is noteworthy to mention that the estimations described above can be further enhanced by including additional variables into the various score functions, such as, but not limited to, variables that take the pharmacological potency of chemical structures into account.

### EXAMPLE NO. 19

### Conducting Similarity Searches

It is clear from the previous examples that the concept of molecular similarity, as viewed by the method of the present invention, is strikingly different from what is generally accepted as being the significance of this term. For example, the compounds in the hypothetical list of example No. 14 are very dissimilar, insofar as there is no obvious way to classify the nine molecules into a single chemical family using classical clustering techniques. Nevertheless, we have shown in example No. 14 that these compounds are, in actual fact, extremely similar, insofar as they each contain at least one occurrence of a chemical determinant that is a representative fragment of the amino acid tyrosine; see this panel:

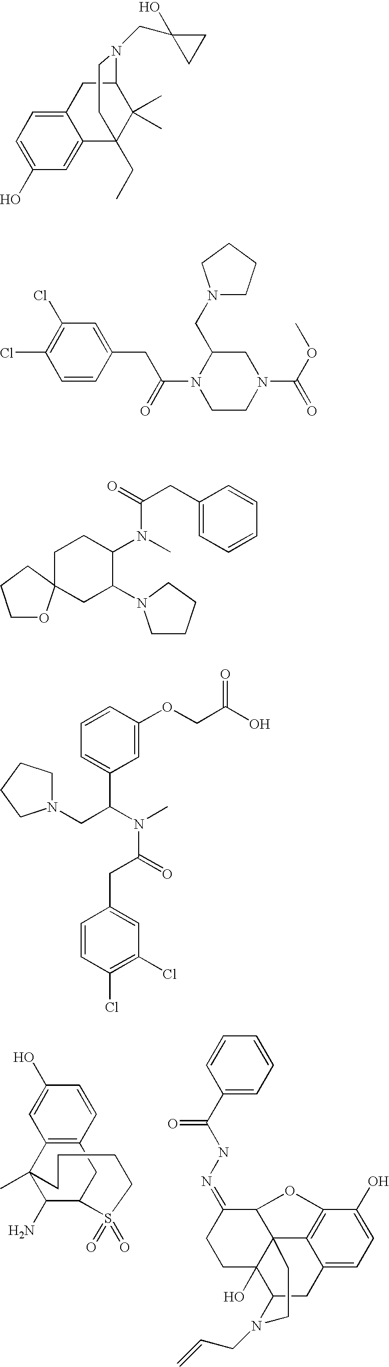

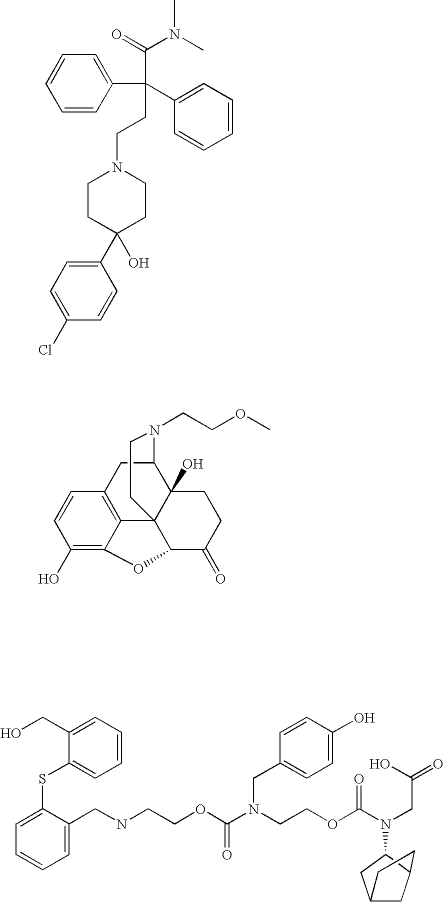

These are fragments of the amino acid tyrosine contained within the structures of nine opiod receptor agonists. The structures shown above are dissimilar insofar as they are difficult to assemble into a single chemical family using classical clustering techniques. They are nevertheless very similar in the sense of the present invention, insofar as they all contain at least one fragment of the chemical determinant defined by the amino acid tyrosine, occurrences of which are highlighted in bold.

As such, the invention can readily be used for measuring molecular similarity and/or for comparing similarities that may exist between different sets of chemical compounds. Illustrating the concept in brief, it is readily apparent that one or more reference molecules can be selected from a list of chemical structures, and analyzed for the presence of certain chemical determinants, which after identification, can be used to conduct one or more substructure searches in one or more new molecules in order to ascertain whether these are similar to the first. By scoring the corresponding chemical determinants with a score function of the type described in the preceding examples, and by scoring the new chemical structures on the basis of, for example, the number of different determinants that they may contain, it is possible to assign values to the molecules being tested which reflect the degree of similarity with the original set of reference compounds. This process is very useful in the design of focused compound collections for drug discovery, as it allows the researcher to rapidly identify compounds bearing large amounts of similarity, in the sense of the present invention, with pharmacologically active reference compounds.

### EXAMPLE NO. 20

### Analyzing the Diversity of Compound Collections

The invention may further be used to analyze the diversity of a compound collection in a manner analogous to that described in the preceding example. In this context, it is apparent to the skilled practitioner that the concept of chemical determinants can readily be used to compare a given compound collection to any other. For example, a collection of compounds can be selected for highthroughput screening by analyzing the the corresponding list of chemical structures according to the invention, wherein a reference set of chemical structures, such as those contained in the Merck Index, Derwent, MDDR or Pharmaprojects databases is used as a reference collection of “drug-like” molecules. In this instance, molecules whose structures are substantially comprised of low scoring chemical determinants are deemed to be “drug-like”, as the same said chemical determinants are present in a high proportion of the reference structures. Conversely, molecules that are substantially comprised of high scoring chemical determinants are deemed to be “non-drug-like”, as the same determinants are only poorly represented within the set of reference compounds. This information is very useful for the design of discovery experiments, as it assists the researcher in identifying chemical structures that should be included or excluded from a compound collection for screening. In this context, it is apparent that a number of algorithms comprised of various combinations of the variables x, y, z and N can be used for this purpose.

### EXAMPLE NO. 21

### Special Algorithms

It is clear that the preceding examples do not supply an exhaustive list of every algorithm employing various combinations of the variables x, y, z and N that can be used for performing discrete substructural analysis. In this context, it is apparent to the skilled practitioner that score functions (XII), (XIII) and (XIV) can also be employed to address a number of the questions presented in the preceding examples. Indeed, in some cases it is even more appropriate, in the statistical sense of the term, to employ one of these formulas instead of the ones explicitly provided in the examples. However, as the invention is primarily designed for identifying the chemical determinants contained within a list of chemical structures that are most likely to be at the basis of a given biological effect, we are primarily concerned with the relative scoring and subsequent rank ordering of chemical determinants. Nevertheless, formulas (XII), (XIII) and (XIV) are supplied below in the event that: a) an exact estimation of the probability of chance occurrence is required for small sample sets (see XII, where s corresponds to the smallest value among the Variables x, (y−x), (z−x) and (N−y−z+x)); b) that a proportionally weighted estimation of the simultaneous contributions of two determinants is felt to be more appropriate for use in example No. 8 (see XIII, where d corresponds to the number of separate chemical determinants); or c) that it is deemed important to estimate order effects when assessing the simultaneous contributions of two interconnected chemical determinants (see XIV). In this context, the definitions of the variables x, y, z and N are exactly those previously described.

$$\begin{matrix}Score = {\sum }_{i = 1}^{s} ( \frac{y ! ( N - y ) ! z ! ( N - z ) !}{x ! ( y - x ) ! ( z - x ) ! ( N - y - z + x ) ! N !} ) & ( XII ) \\ Score = {\sum }_{i = 1}^{d} {( \frac{Nx - yz}{N} )}_{i} / \sqrt{{\sum }_{i = 1}^{d} {( \frac{z ( N - z ) y ( N - y )}{{N}^{3}} )}_{i}} & ( XIII ) \\ Score = \frac{{( \vert y + z - N \vert - 1 )}^{2}}{( N - y - z + 2 x )} & ( XIV )\end{matrix}$$

Finally, it is also apparent to the skilled practitioner that the use of certain variables in score functions and/or algorithms designed to identify biologically active chemical determinants, but not explicitly described in the preceding examples, can be mathematically equivalent to using various combinations of the variables x, y, z and N. Illustrating this, a score function employing the variable q, defined as representing the number of inactive molecules whose chemical structures contain a given chemical determinant, is equivalent to employing x and y, as q=y−x. Likewise, a score function employing the variable r, defined as representing the total number of active compounds that do not contain a given chemical determinant, is algebraically equivalent to employing the variables x and z, as it can readily be shown that r=z−x. Also, a score function employing a variable s, defined as representing the total number of inactive compounds that do not contain a given chemical determinant, is equivalent to employing the variables x, y, z and N, as s=N−y−z+x. Finally, algorithms employing the variables t and u, respectively representing the total number of molecules whose structures do not contain a given determinant (t), and the total number of inactive molecules (u), are equivalent to employing the variables N, y and/or z, as it can readily be shown that t=N−y, and u=N−z.

### EXAMPLE NO. 22

### Mapping Relative Contributions

The invention also allows for the construction of relative contribution diagrams. These are graphical representations of chemical structures where the relative contribution of various atoms, bonds, fragments and/or substructures to a given biological outcome are indicated by score values calculated as described in the preceding examples. In a preferred embodiment of the method, probabilistic score values such as those calculated using formula (XII) are used, where P(A) represents the probability that a given chemical determinant is contained within the subset of biologically active structures on the basis of random chance, which is calculated using formulae employing various combinations of the variables x, y, z and N as previously described.

Score=[1−P(A)]·100% (XII)

In this context, it is evident that numerous measures of association and/or score functions can be used to estimate P(A). Two examples of relative contribution diagrams will now be discussed in more detail. The following panel

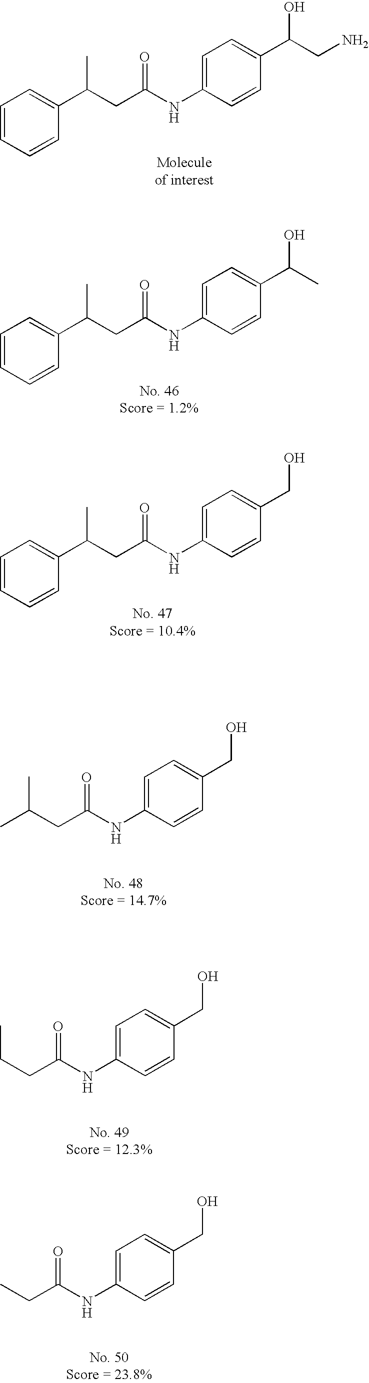

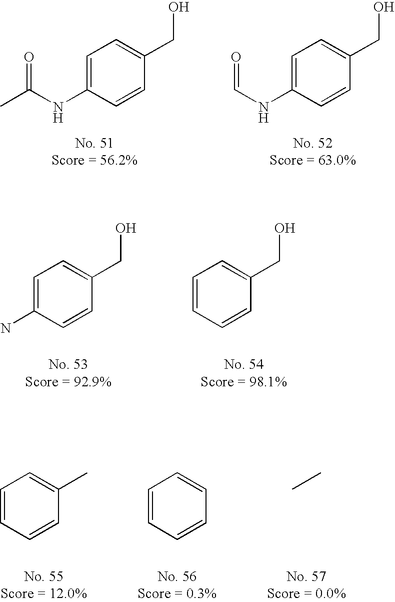

shows a molecule of interest accompanied by a series of chemical determinants comprised of fragments of the same said molecule, that were scored using formula (XII) and a modification of measure of association (I) to determine P(A). FIG. 15 shows the same information in graphical form, where the determinants are plotted versus their respective score values. In this context, it is apparent that the same information can be represented in the form of probabilistic contour maps, as shown in this panel:

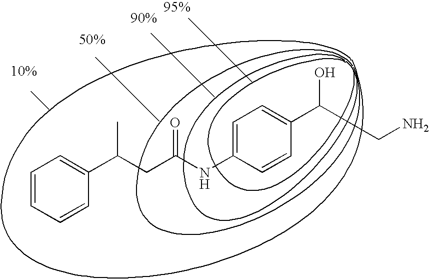

Overall, such diagrams are very useful for designing compound collections, as they assist the researcher in selecting compounds on the basis of mathematical estimations of the chance of being successful in a given assay, reducing the need to rely on the concept of molecular diversity to identify novel, biologically active chemical series. They are also of interest in medicinal chemistry, as representations such as the one shown in the above panel, clearly indicate which moieties of a molecule can reasonably be modified with minimal risk of loosing pharmacological activity. Conversely, such graphs alert the toxicologist as to which moieties of a toxic compound need to be modified in order to eliminate an undesirable effect.

For obtaining the relative contribution mappings shown above and in FIG. 15, chemical determinants corresponding to fragments of a biologically active molecule were scored according the invention using a score function employing the variables x, y, z and N that permitted a direct estimation of the probability of chance occurrence within the set of active molecules (P(A)). The corresponding P(A) values were transformed using score function (XII), supplying a probabilistic score value for each determinant reflecting the relative likelihood that the corresponding chemical structure was at the basis of the biological activity of interest. The values can be illustrated as in FIG. 15 which is a graphical representation of the score values for the various chemical determinants. Chemical determinant No. 54 corresponds to the local maximum of this series. Or, the values can be illustrated as in the above panel which is a probabilistic contour map, indicating which fragment or sector of the chemical structure of interest is most likely to confer biological activity (determinant No. 54 contained within the area delimited by the 95% contour line). Another way of presenting the values in shown in FIG. 11.

### EXAMPLE NO. 23

### Equivalence of Score Functions

The score functions employed in the previous examples are all ways to identify chemical determinants that are most likely to be at the basis of a given biological, pharmacological and/or toxicological effect. Whilst it is apparent to the skilled practitioner that certain measures of association and/or score functions are best used for addressing only certain types of question, when employed as described in the method of the present invention, each formula allows for the identification of the same, highest ranking chemical determinant that is most likely to be at the basis of a given biological effect. As such, the formula presented in the preceding examples are functionally equivalent in the sense of discrete substructural analysis.

Demonstrating this, an analysis of the chemical structures of 131 dopamine D₂ receptor agonists was performed eight times in parallel using the eight measures of association and score functions containing various combinations of the variables x, y, z and N shown below. The study was conducted as previously described, notably by adding the chemical structures of 101207 molecules described as having no effect on the dopamine D₂ receptor to the first list of 131, and scoring the series of 19 chemical determinants shown below with score functions (XV) to (XXIII), which the reader will recognize as representing the same functions that were employed in a number of previous examples, and/or closely related variants thereof.

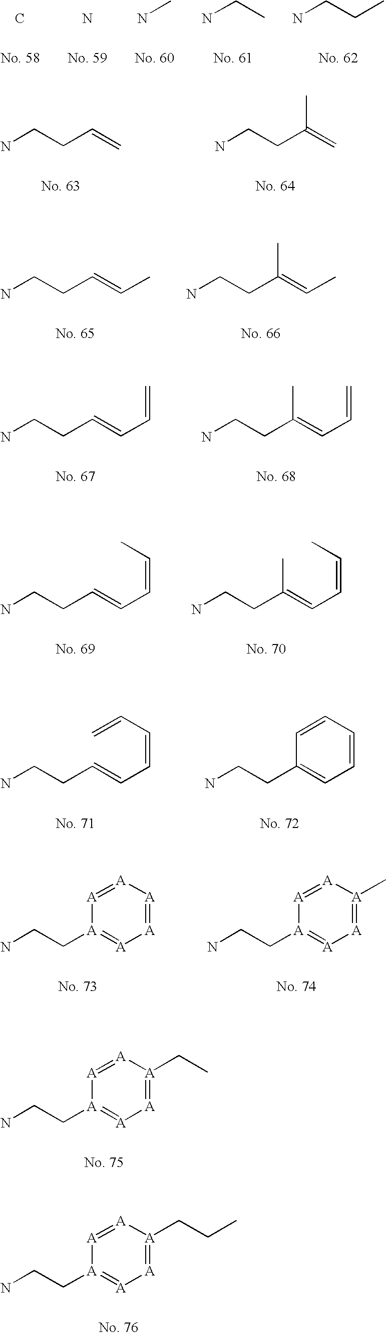

These are the chemical determinants scored with eight different score functions. The 19 chemical determinants shown above were scored using functions (XV) to (XXII) and a list of chemical structures annotated for dopamine D₂ receptor agonist activity. The used functions are:

Score=MW·(x/z) (XV)

Score=(x/z)−(y/N) (XVI)

Score=Nx−yz (XVII)

$$\begin{matrix}Score = \frac{x ( N - y - z - x )}{( z - x ) ( y - x )} & ( XVIII ) \\ Score = \frac{{( \vert Nx - yz \vert - N / 2 )}^{2} N}{z ( N - z ) y ( N - y )} & ( XIX ) \\ Score = \frac{x ( N - y - z - x )}{( z - x ) ( y - x )} {ⅇ}^{- 2 \sqrt{1 / x + 1 / ( y - x ) + 1 / ( z - x ) + 1 / ( N - y - z + x )}} & ( XX ) \\ Score = \frac{Nx - yz}{\sqrt{z ( N - z ) y ( N - y )}} & ( XXI )\end{matrix}$$

Score=e^{[(x/z)-(z-x)/(N-z)]} (XXII)

FIGS. 16A to 16H show corresponding relative contribution diagrams. The chemical determinants shown in the above panel were scored as previously described, and plotted versus their corresponding score values. FIG. 16A shows the scores obtained with function (XV), FIG. 16B the scores obtained with function (XVI), FIG. 16C the scores obtained with function (XVII), FIG. 16D the scores obtained with function (XVIII), FIG. 16E the scores obtained with function (XIX), FIG. 16F the scores obtained with function (XX), FIG. 16G the scores obtained with function (XXI), and FIG. 16H the scores obtained with function (XXII). Each score function invariably singled out the same chemical determinant (No. 73) as being the most likely to be at the basis of biological activity.

As shown by the relative contribution diagrams presented in FIGS. 16A to 16H, each of the eight score functions correctly identified chemical determinant No. 73 as corresponding to a local maximum, signifying that it is the chemical motif most likely to be at the basis of dopamine D₂ agonist activity within the list of 19 tested determinants. Interestingly, the different score functions varied in terms of ranking lower-scoring chemical determinants, insofar as determinant No. 62 was suggested as being of importance to biological activity by ranking third in calculations using score functions (XV), (XVI) and (XVII), whereas determinant No. 63 ranked third using score function (XXII), determinant No. 65 ranked third according to score functions (XIX) and (XXI), and finally, determinant No. 66 ranked third when tested with score functions (XVIII) and (XXII).

Overall, these minor differences are of little importance to the successful outcome of the method, as in each case, the lower ranking determinants are actually fragments of the larger, highest ranking determinant No. 73 (see the above panel). As such, it suffices to directly employ chemical determinant No. 73 and fragments thereof for the design of compound collections for highthroughput screening, as these will invariably contain structures containing of each of the lower ranking determinants. A sampling of the type of compound that could to included into such a collection is shown below.

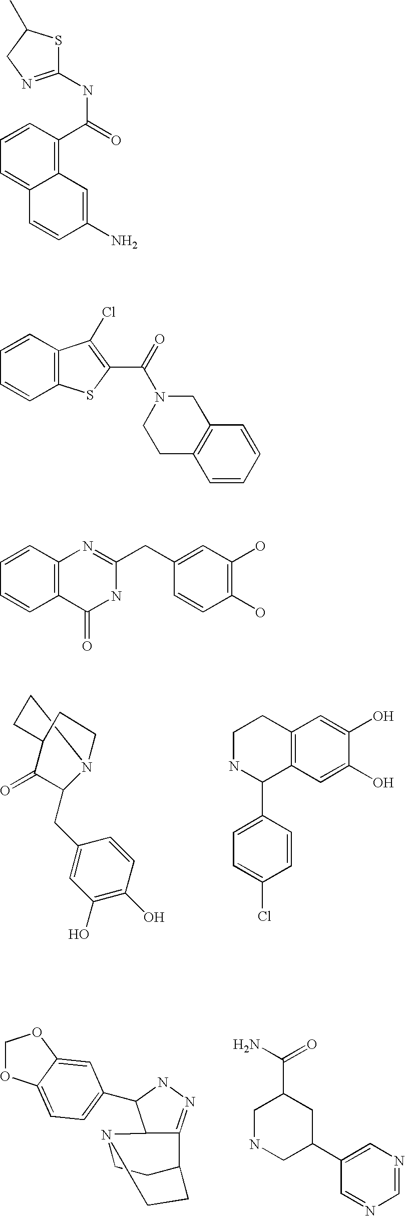

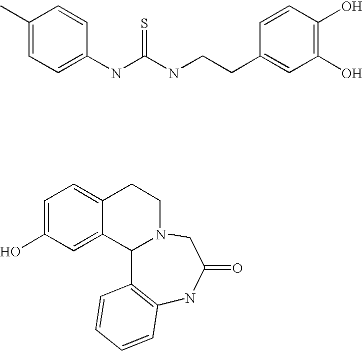

These sample structures are examples of compounds that could be selected for inclusion into a compound collection designed for the identification of dopamine D₂ receptor agonists. Each of the structures shown above contains a chemical determinant No. 73, or a substantial portion thereof.

In conclusion, and whilst the mathematical reasoning lying behind the construction and use of the eight different score functions is different in each case, all of these identify the very same chemical determinant that is most likely to be at the basis of biological activity. As such, algorithms containing various combinations of the variables x, y, z and N, or q, r, s, t and u as previously mentioned, are functionally equivalent in the sense of the present invention.

### EXAMPLE NO. 24

### Informatics-Based Tools for Drug Discovery

It is apparent from the preceding examples that the present invention can be incorporated into one or more series of procedures, such as, but not limited to, computer programs designed to increase the efficiency of highthroughput screening, compound discovery, hits-to-leads chemistry, compound progression and/or lead optimization. Such procedures or programs are preferably be designed to direct machines and/or robotic systems that perform drug screening, compound selection, set generation, and/or chemical synthesis in a supervised, semi-autonomous, or fully autonomous manner. Such procedures comprise, but are in no way limited to, the following examples which form preferred embodiments of the present invention:

A process whereby chemical structures, annotated with corresponding experimental results, are analyzed, and biologically active chemical determinants are identified according to the invention.

A process whereby biologically-active chemical determinants identified according to the invention are used to conduct searches in chemical databases, virtual or other, in order to identify compounds, biologicals, reagents, reaction products, intermediates or other, that are most likely to exhibit a given pharmacological, biochemical, toxicological and/or biological property.

A process whereby biologically active chemical determinants identified according to the invention are stored in a register along with accompanying experimental data and/or score values, in an electronic form or other, and regularly updated or not, which serves as a repository of structural information for use in a decision making process, automated or not, for chemical compound, series and/or scaffold selection for highthroughput screening, medicinal chemistry and/or lead optimization, said experimental results and score values relating to any given pharmacological, biochemical, toxicological and/or biological property.

A process whereby the invention, as described in any of the preceding examples, is used for the identification of pharmacological modulators of drug targets, such as for example, but not limited to, receptor ligands, kinase inhibitors, ion channel modulators, protease inhibitors, phosphatase inhibitors and steroid receptor ligands.

A process whereby the invention, as described in any of the preceding examples, is directly used, or employed in a computer program designed to analyze chemical structures in order to increase the potency of a chemical series, increase the selectivity of a chemical series, design compounds with multiple pharmacological effects, predict the potential secondary pharmacological actions of a molecule, predict the potential toxicological actions of a molecule, identify the biologically active moieties of receptor ligands, predict potential protein-protein interactions, identify orphan ligand-receptor pairs, and/or identify endogenous modulators of drug targets. The latter uses refer in particular to the fields of functional genomics and proteomics, wherein, for example, nucleotide and/or amino acid sequences can be selected for investigation on the basis of the chemical structures of molecules identified in a biochemical screening assay and processed according to the invention, such as, for example, for the identification of orphan ligands.

A process whereby the invention is either directly used, or used in programs designed to identify false positive and/or negative experimental results.

A process whereby the invention is either directly used, or used in programs designed to predict the potentially hazardous effects of a molecule to man, livestock and/or the environment, such as, for example, in the screening of chemicals for use in or as food additives, in plastics, textiles, and the like.

A process whereby the invention is either directly used, or used in a program designed to perform configurational, conformational, stereochemical, similarity and/or diversity analyses.

A process whereby the invention is either directly used, or used in a program designed to generate relative contribution maps and/or graphical representations of the biologically active moieties or chemical structures.

A process whereby any of the processes outlined above, employed alone or in either serial and/or parallel combinations, are used for the functioning of an informatics tool, computer program, and/or expert system intended for use in the conduct of drug, herbicide, and/or pesticide discovery.

A process whereby any of the processes outlined above, employed alone or in serial and/or parallel combinations, are used for directing the function of machinery and/or instrumentation, automated or not, autonomous or not, and using updatable registers of chemical determinants annotated with score values or not, for use in the rational generation of chemical structures, the retrieval of chemical compounds, the rational generation of experimental protocols and/or screening data, and/or the rational selection of results and/or chemical structures in the pharmaceutical and/or agricultural discovery sectors.

Other procedures of incorporating the invention are easily obtainable by means of the skilled person's common knowledge.

1. Method of operating a computer system to perform a discrete substructural analysis, the method comprising the steps of:
accessing (210, 220, 410) a database (110, 115) of molecular structures, the database being searchable by molecular structure information and biological and/or chemical properties;
identifying (220) in said database a subset of molecules having a given biological and/or chemical property;
determining (230, 420) fragments of the molecules in said subset;
for each fragment, calculating (230, 430, 610-650) a score value indicating the contribution of the respective fragment to said given biological and/or chemical property; and
performing (240, 250) a reiteration process by analyzing (250) the determined fragments and calculated score values, whereby first at least one fragment is selected that has a score value indicating high contribution to said biological and/or chemical property, and then repeating the steps of accessing, identifying, determining and calculating.

2. The method of claim 1, wherein the step of calculating a score value includes the step of:
calculating (610) the number of molecules (x) within said subset of molecules that contain a given fragment.

3. The method of one of claims 1 or 2, further comprising the step of:
identifying in said database a second subset of molecules not having said biological and/or chemical property;
wherein said step of calculating a score value comprises the step of:
calculating (620) the number of molecules (y) within said subset and said second subset of molecules that contain a given fragment.

4. The method of one of claims 1 to 3, wherein said step of calculating a score value comprises the step of:
calculating (630) the number of molecules (z) within said subset of molecules.

5. The method of one of claims 1 to 4, further comprising the step of:
identifying in said database a second subset of molecules not having said given biological and/or chemical property;
wherein said step of calculating a score value comprises the step of:
calculating (640) the total number of molecules (N) within said subset and said second subset of molecules.

6. The method of one of claims 1 to 5, wherein the reiteration process is performed by chosing the fragments of the next round to be of higher molecular weight than the fragments of the previous round.

7. The method of one of claims 1 to 6, further comprising the steps of:
selecting (710) a fragment based on the calculated score values;
analyzing (810) the structure of the selected fragment;
locating (820) a generalized item in the fragment structure; and
replacing (830) the generalized item with a generalized expression to generate a generic substructure.

8. The method of claim 7, further comprising the step of:
performing (840) a virtual screening using the generic substructure.

9. The method of one of claims 1 to 8, wherein the step of analyzing the determined fragments and the calculated score values comprises the steps of:
selecting (1010) a first fragment based on the calculated score values;
selecting (1020) a second fragment based on the calculated score values; and
generating (1030) a molecular substructure including said first fragment and said second fragment by applying an annealing function.

10. The method of one of claims 1 to 9, wherein the step of analyzing the determined fragments and calculated score values comprises the steps of:
selecting (710) at least one fragment based on the calculated score value;
extracting (720) compounds from the previous subset of molecules, the extracted compounds containing the selected fragment;
selecting (730) compounds from the previous subset of molecules not containing the selected fragment, or compounds not included in the previous subset of molecules; and
forming (740) a new subset of molecules including the extracted and the selected compounds.

11. The method of one of claims 1 to 10, further comprising the step of:
generating (230) a fragment library (120) including the determined fragments and the calculated score values.

12. The method of one of claims 1 to 11, wherein said database is a proprietary database.

13. The method of one of claims 1 to 12, wherein said database is a public database.

14. The method of one of claims 1 to 13, wherein said database is a database of amino acid and/or nucleic acid sequences, and said biological and/or chemical property is a given effect on a protein of interest.

15. The method of one of claims 1 to 14, wherein said biological and/or chemical property is a pharmacological property, and the method is used for drug discovery.

16. The method of one of claims 1 to 15, further comprising the step of:
compiling (260) a set of compounds that contain at least one of the determined fragments.

17. The method of claim 16, further comprising the step of:
testing the compounds of said compiled set for said given biological and/or chemical property.

18. Computer program product arranged for performing the method of one claims 1 to 17.

19. Fragment library generated by performing the method of one of claims 1 to 17.

20. Computer system for performing a discrete substructural analysis, comprising;
means (100, 110, 115) for accessing a database of molecular structures, the database being searchable by molecular structure information and biological and/or chemical properties;
means (100, 130) for identifying in said database a subset of molecules having a given biological and/or chemical property;
means (100, 130, 135) for determining fragments of the molecules in said subset;
means (100, 130, 140) for calculating, for each fragment, a score value indicating the contribution of the respective fragment to said given biological and/or chemical property; and
means (100, 130) for determining whether a reiteration is to be performed, and if so, analyzing the determined fragments and calculated score values, and performing a reiteration process.

21. The computer system of claim 20, arranged for performing the method of one of claims 1 to 17.

22. Drug compound obtained by synthesising a molecule containing at least one fragment determined by performing the method of one of claims 1 to 17.
